(19) United States
(12) Patent Application Publication
(10) Pub. No.: US 2026/0124273 A1
(43) Pub. Date: May 7, 2026

(54) Composition, Method, And Use of Netrin-1 In Preserving the Bone Marrow Niche to Promote Stem Cell Health

(71) Applicant: Hackensack Meridian Health, Inc., Edison, NJ (US)

(72) Inventors: Jason Mathew Butler, New York, NY (US); Pradeep Ramalingam, Edison, NJ (US)

(73) Assignee: Hackensack Meridian Health, Inc., Edison, NJ (US)

(21) Appl. No.: 19/117,542

(22) PCT Filed: Oct. 3, 2023
(86) PCT No.: PCT/US2023/034334

§ 371 Date: Apr. 1, 2025

Related U.S. Application Data
(60) Provisional application No. 63/413,033, filed on Oct. 4, 2022.

(51) Int. Cl. A61K 38/17; A61P 7/00; A61P 35/02

(52) U.S. Cl. CPC A61K 38/1709; A61P 7/00; A61P 35/02

(57) ABSTRACT

Disclosed is a method of using Netrin-1 (NTN1) driven rejuvenation of an aged hematopoietic system based on its shown dependency of reestablishing integrity of aged bone marrow niche and reactivating DNA damage response (DDR). NTN1 is shown as a master regulator of reactivating DNA damage response (DDR) pathways. Every organ/tissue accumulates DNA damage that is an underlying cause of diseases. Reactivating DDR has an extensive effect on treating diseases. NTN1 serves as a therapeutic modality that effectively reverses age-related hematopoietic deficiencies while simultaneously targeting growth and survival of acute myelogenous leukemia (AML). The ability to target and gene correct hematopoietic stem cells (HSC) ex vivo for the treatment of hemoglobinopathies, like thalassemia and sickle cell anemia, is limited due to inability of transducing the correct cell population. NTN1 is shown to maintain and expand bona fide HSCs, opening up the possibility to transduce a true stem cell overcoming previous limitations.

### CROSS-REFERENCE TO RELATED APPLICATIONS

The present application is a U.S. national phase entry under 35 U.S.C. § 371 of International Application No. PCT/US2023/34334, filed, Oct. 3, 2023 which claims the benefit of the filing date of U.S. Provisional Application No. 63/413,033, filed Oct. 4, 2022, entitled Composition, Method, And Use of Netrin-1 In Preserving the Bone Marrow Niche to Promote Stem Cell Health, the disclosure of which is hereby incorporated herein by reference.

### TECHNICAL FIELD

The present disclosure relates to a method, composition, and uses of Netrin-1 and application thereof in rejuvenating aged niche cells and restoring blood stem cell function.

The present disclosure also relates to a method composition, and uses of Netrin-1 in maintaining an active DNA damage response (DDR) and method for reversing functional deficits within aged blood stem cells by rejuvenating their supportive niche within bone marrow.

### BACKGROUND

The human body is susceptible to disease every day. Each of the approximately ten trillion cells in the human body receives tens of thousands of DNA lesions per day. These lesions can block genome replication and transcription, and if they are not repaired or are repaired incorrectly, they lead to mutations or wider-scale genome aberrations that threaten cell or organism viability. Some DNA aberrations arise by physiological processes, such as DNA mismatches occasionally introduced during DNA replication and DNA strand breaks caused by abortive topoisomerase I and topoisomerase II activity.

In addition, hydrolytic reactions and non-enzymatic methylations generate thousands of DNA-base lesions per cell per day. DNA damage is also produced by reactive-oxygen compounds arising as by-products from oxidative respiration or through redox-cycling events involving environmental toxins and Fenton reactions mediated by heavy metals. Reactive oxygen and nitrogen compounds are also produced by macrophages and neutrophils at sites of inflammation and infections. Such chemicals can attack DNA, leading to adducts that impair base-pairing and/or block DNA replication and transcription, base loss, or DNA single-strand breaks (SSBs). Furthermore, when two SSBs arise in close proximity, or when the DNA-replication apparatus encounters a SSB or certain other lesions, double-strand breaks (DSBs) are formed. While DSBs do not occur as frequently as the other lesions listed above, they are difficult to repair and extremely toxic.

Today, probably the most prevalent environmental cancer-causing chemicals are those produced by tobacco products, which trigger various cancers, most notably those of the lung, oral cavity, and adjacent tissues. Cancer-causing DNA-damaging chemicals can also contaminate foods, such as aflatoxins found in contaminated peanuts and heterocyclic amines in over-cooked meats.

To combat threats posed by DNA damage, human cells have mechanisms collectively termed the DNA-damage response (DDR)-to detect DNA lesions, signal their presence and promote their repair. Cells defective in these mechanisms generally display heightened sensitivity towards DNA-damaging agents, and many such defects cause human disease. Although responses differ for different classes of DNA lesions, they usually occur by a common general regime.

The issue is DDR in cells, especially in aged systems, do not function properly and fail to do the repairs needed thus resulting in a disease state. There is an urgent need to discover how to promote DDR in cells.

Netrins are known to regulate neuronal migration, axon guidance and synaptogenesis in the nervous system. Netrins are a class of laminin-like proteins, which were first identified as axonal guidance cues during embryonic development. Netrin-1 protein is membrane associated in the adult spinal cord. Over recent years, it has become apparent that netrin-1 may additionally be involved in the underlying pathology of several multisystem diseases, making it an attractive potential therapeutic target. However, there are conflicting reports as to whether netrin-1 acts in a pro- or anti-angiogenic capacity. For example, in atherosclerosis, opposing effects have been reported on plaque progression, due to the ability of netrin-1 to inhibit both macrophage egress from and monocyte ingress into lesions.

Endothelial netrin-1 expression has also been shown to regulate leucocyte migration, and modulation of this could potentially provide benefit in exacerbating or attenuating inflammatory responses. To date, there has been limited research on other proteins in the netrin family, which may also be involved in these processes. Even in the case of netrin-1, further investigation is needed to determine whether it can play a part in the prevention of cardiovascular disease as well as other inflammatory pathologies. The seemingly contradictory actions of netrin-1 in certain physiological pathways, such as angiogenesis, serve to further highlight its intricate and multiple roles in numerous disease processes. Whether inhibition or augmentation of netrin-1 expression will provide clinical benefit in different pathophysiological situations remains to be seen.

Therefore, there is a need to investigate the use of netrin-1 and its effect on certain disease states. There is a need for an improved regime for using netrin-1, effective composition, and dosage of use of netrin-1.

### SUMMARY

Compared to the above prior attempts, the present disclosure fulfills the above criteria and provides additional benefits that state of the art systems cannot provide.

Stem cells reside in specialized tissue-specific microenvironments termed ‘niches’ wherein they receive instructive signals to preserve their regenerative potential. Aging associated defects within stem cell-supportive niches contribute towards age-related decline in stem cell function. However, the causes underlying age-related niche defects and whether restoring niche function can improve stem cell fitness during aging, remain unclear.

The present investigator sought to determine whether functional deficits within aged blood stem cells can be reversed by rejuvenating their supportive niche within the bone marrow. It was identified herein a critical regulator of niche cell fitness during homeostasis, regeneration, and aging. Deletion of this factor in young mice induces premature aging of the bone marrow niche, while supplementation of aged mice rejuvenates aged niche cells and restores blood stem cell function to youthful levels.

It is further shown herein that this niche-derived signal plays an essential role in maintaining an active DNA damage response (DDR) and that loss of this niche-derived factor results in accumulation of DNA damage within the bone marrow niche and blood stem cells.

The present investigator shows that supplementation with this critical regulator during aging is sufficient to reactivate DDR, resolve DNA damage, and restore functional potential within the aged bone marrow niche and blood stem cells and can restore the regenerative capacity of an aged hematopoietic system to endure serial chemotherapy regimens.

Accumulation of DNA damage is a hallmark feature of aging and widely considered the central cause of the aging process. However, identification of master regulators of DNA repair that affects multiple DDR pathways and development of strategies to reactivate the DNA Damage Response (DDR) in aging tissues is lacking and is currently a major focus in aging research. The present investigator has identified the Netrin-1 (NTN1) signaling pathway is selectively expressed by vascular endothelium (VE) and mesenchymal stem cells (MSCs) to maintain aged hematopoietic stem cells (HSCs). The present investigator demonstrates herein that systemic infusion of NTN1 restores aged HSC function by reactivating the DDR.

Additionally, it was found by the present investigator that infusion of NTN1 reactivated the DDR to promote rapid hematopoietic recovery, improved self-renewal capacity, increased longevity, and reduces weight loss following single and serial myelosuppressive insults. Moreover, it was demonstrated by the present investigator herein that treatment of acute myeloid leukemia (AML) with NTN1 results in a decrease in the expansion of AML cells due to apoptosis.

The data presented in this disclosure demonstrates that that NTN1 antagonizes leukemia by maintaining aged VE and MSC integrity, gives a competitive advantage to aged HSCs, while eradicating AML simultaneously. Lastly, it was demonstrated by the present investigator that NTN1 can be supplemented in ex vivo expansion protocols to maintain and enhance the function of expanded young and aged HSCs, opening up the door for gene correction of hemaglobinopathies.

DDR within stem cells is presumed to be regulated cell-intrinsically, in intricate co-ordination with cell-cycle decisions. It has been suggested that predominantly quiescent aged HSCs require cell-cycle activation to repair their accumulated DNA damage.

However, the present investigate based on the findings herein dispel these long-standing paradigms and demonstrate for the first time that HSCs depend on niche-derived signals to activate their intrinsic DDR. Given that impaired DDR is the root cause for a diverse array of intractable pathophysiological aging conditions, the findings from this study will have tremendous implications for diverse fields within the scientific community.

Moreover, the potential of NTN1 infusion to rejuvenate aged HSCs and their niche and its potential to restore regenerative capacity of an aged hematopoietic system to withstand serial chemotherapy will have immediate therapeutic impact.

### BRIEF DESCRIPTION OF THE DRAWINGS

The patent or application file contains at least one drawing executed in color.

Copies of this patent or patent application publication with color drawing(s) will be provided by the Office upon request and payment of the necessary fee.

To assist those of skill in the art in making and using the disclosed composition and method, reference is made to the accompanying figures, wherein:

FIGS. 1a-1g illustrate loss of netrin-1 (NTN1) induces premature ageing of the BM niche;

FIGS. 2a-2j illustrate NTN1 supplementation rejuvenates the aged BM niche by reactivating their DDR;

FIGS. 3a-3o show NTN1 rejuvenates aged HSCs and restores their regenerative capacity;

FIGS. 4a-4o illustrate NTN1 reactivates the dampened DDR within aged HSCs;

FIGS. 5a-5g show Extended Data 1 where NTN1 is expressed by LepR+ MSCs and BMECs within the BM;

FIGS. 6a-6j show Extended Data 2 illustrating MSC derived NTN1 is essential for maintaining HSC homeostasis;

FIGS. 7a-7j show Extended Data 3 illustrating Loss of EC-derived NTN1 causes premature HSC aging phenotypes;

FIGS. 8a-8j show Extended Data 4 illustrating Niche derived NTN1 preserves HSC fitness during aging;

FIGS. 9a-9j show Extended Data 5 illustrating NTN1 regulates the DDR within LepR+ MSCs;

FIGS. 10a-10j show Extended Data 6 illustrating NTN1 regulates the DDR within BMECs;

FIGS. 11a-11d show Extended Data 7 illustrating NTN1 revitalizes the aged BM vascular niche;

FIGS. 12a-12d show Extended Data 8 illustrating NTN1 accelerates hematopoietic recovery in aged mice;

FIGS. 13a-13f show Extended Data 9 illustrating DDR downregulation is a conserved feature of HSC aging;

FIGS. 14a-14g show Extended Data 10 illustrating NTN1 is sufficient to rejuvenate aged HSCs;

FIGS. 15a-15e show Netrin-1 supplementation promotes the maintenance and function of ex vivo expanded adult HSCs; and

FIGS. 16a-16g show Aging and NTN1 signaling regulates AML progression.

### DETAILED DESCRIPTION

The invention includes, according to certain embodiments, compositions, uses, and processes relating to Netrin-1 (NTN1) in maintaining an active DNA damage response (DDR) and method for reversing functional deficits within aged blood stem cells by rejuvenating their supportive niche within bone marrow.

Stem cells reside in specialized tissue-specific microenvironments termed ‘niches’ wherein they receive instructive signals to preserve their regenerative potential. Aging associated defects within stem cell-supportive niches contribute towards age-related decline in stem cell function^{1,2}. However, the causes underlying age-related niche defects, and whether restoring niche function can improve stem cell fitness during aging, remain unclear.

In this investigation, it was sought to determine whether functional deficits within aged blood stem cells can be reversed by rejuvenating their supportive niche within the bone marrow. It was identified herein that Netrin-1(NTN1) is a critical regulator of niche cell fitness during homeostasis, regeneration, and aging. Deletion of Netrin-1 in young mice induces premature aging of the bone marrow niche, while supplementation of aged mice with Netrin-1 rejuvenates aged niche cells and restores blood stem cell function to youthful levels. It was also shown herein that Netrin-1 plays an essential role in maintaining an active DNA damage response (DDR) and that loss of niche-derived Netrin-1 results in accumulation of DNA damage within the bone marrow niche and blood stem cells.

It was identified herein that DDR downregulation and DNA damage accumulation represent fundamentally conserved attributes of aged blood stem cells and their niche. Netrin-1 supplementation during aging is sufficient to reactivate DDR, resolve DNA damage, and restore functional potential within the aged bone marrow niche and blood stem cells. Lastly, the present investigator demonstrated herein that Netrin-1 mediated rejuvenation of aged blood stem cells and their niche is sufficient to restore the regenerative capacity of an aged hematopoietic system to endure serial chemotherapy regimens.

### Introduction

Aging is associated with an increased risk for hematologic malignancies that frequently require hematopoietic stem cell transplantation (HSCT) therapies. Reduced intensity conditioning (RIC) regimens for achieving myelosuppression essential for HSCTs have greatly expanded the eligibility for HSCT in elderly patients and it is now estimated that ˜39% of allogenic and ˜55% of autologous HSCTs are performed in adults >60 years of age 3,4. While RIC regimens decrease transplant related mortality (TRM), they are associated with an increased risk of relapse4. Contrarily, higher intensity conditioning regimens that improve disease free survival are associated with increased TRM in elderly patients. One of the primary causes that predisposes older patients to a higher risk of negative HSCT outcomes/failures is that aging diminishes the regenerative ability of the hematopoietic system to recover from cytotoxic side effects of myelosuppression, arising in part from an impairment in the fitness and functionality of the HSC-supportive bone marrow (BM) niche5,6. While the critical role played by niche cells in maintaining HSC fitness has been well-established7, there is little insight into the mechanisms underlying age-related deterioration in the HSC-supportive niche activity1,8.

To identify candidate factors that can improve niche function and HSC fitness during aging, we surveyed the transcriptomic data of a recently described murine model that manifests premature aging of their hematopoietic system9. The present analysis revealed a putative role for Netrin-1 (NTN1) in regulating aging of the BM niche. NTN1 is an established axon-guidance cue that has recently been shown to regulate diverse processes including angiogenesis and osteogenesis10-12. In the hematopoietic system, NTN1 was recently identified as a ligand for Neogenin-1 (NEO1) on HSCs, and it has been proposed that BM niche-derived NTN1 engages NEO1 on HSCs to preserve HSC dormancy in young Mice13,14.

However, whether NTN1 regulates BM niche function is unknown. Here the present investigator identified NTN1 as a critical regulator of niche cell fitness during homeostasis, regeneration, and aging. Defined was BM mesenchymal stem cells (MSCs) and BM endothelial cells (BMECs) as the principal sources of niche-derived NTN1 within the BM and demonstrate that NTN1 regulates both niche-HSC and niche-niche interactions. Conditional deletion of NTN1, either in Leptin Receptor+ (LepR+) BM MSCs or ECs of young mice, induces premature aging of their BM niche and hematopoietic system. Adult hematopoietic stem cells (HSCs) reside in a perivascular niche in the bone marrow in which Leptin Receptor⁺ (LepR⁺) perivascular stromal cells and endothelial cells secrete factors that promote their maintenance.

As shown herein is that niche cells and HSCs require NTN1 for maintaining an active DNA damage response (DDR), preventing DNA damage accrual, and preserving functional potential during aging. It was identified herein that a dampened DDR is a fundamentally conserved attribute of EC, MSC, and HSC aging and demonstrate that NTN1 supplementation reactivates the dampened DDR and resolves the accrued DNA damage within niche cells and HSCs.

It is also shown herein that treatment of aged mice with recombinant NTN1 is sufficient to reverse phenotypic defects of the aged BM vascular niche, including improved BM vascular integrity and suppression of age-associated BM adiposity.

Additionally, it is demonstrated herein that NTN1 mediated niche rejuvenation is associated with a restoration of self-renewal capacity of aged HSCs to youthful levels. The beneficial effects of NTN1 on the aged hematopoietic system translates to an accelerated hematopoietic recovery, increased survival, and preservation of body weight during serial myelosuppressive chemotherapy.

In summary, the findings discussed herein indicate that NTN1 supplementation can serve as a therapeutic strategy to enhance integrity of the aged BM vascular niche, restore the functional potential of aged HSCs, and improve survival following myelosuppressive regimens in the elderly population.

### Results

### Loss of NTN1 Induces Premature Aging of the BM Niche.

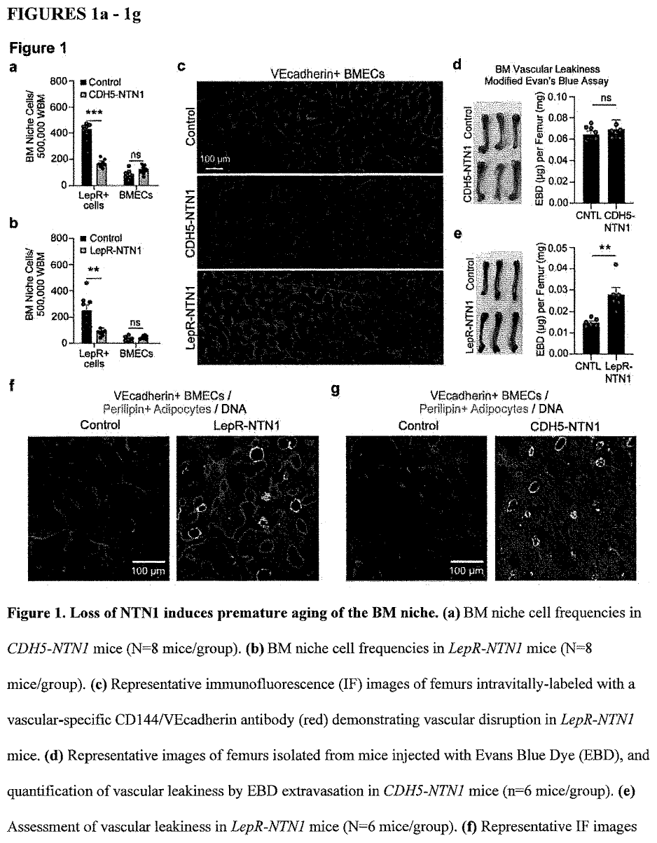

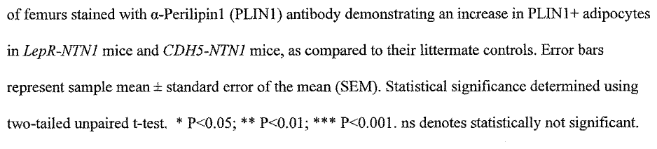

To identify cellular sources of NTN1 within the BM niche, we analyzed a recently published RNA-Seq dataset on BM niche cell subpopulations15. We observed that NTN1 was primarily expressed in BM MSCs with detectable expression in BMECs and that aging is associated with a significant decline in MSC-derived Netrin-1 expression (Extended Data FIG. 1a). It was confirmed NTN1 expression in BM MSCs and BMECs via immunofluorescence analysis of ex vivo cell-cultures and in vivo femoral sections (Extended Data FIG. 1b, c).

To determine whether NTN1 derived from BM MSCs and BMECs regulates BM niche function, we conditionally deleted NTN1 in MSCs and ECs of young adult mice and analyzed their niche parameters (FIG. 1). To delete NTN1 in MSCs, Netrinfl/fl mice16 were crossed with LepR-Cre mice17 to generate LepR−NTN1 mice. To assess the role of EC-derived NTN1 on niche function, we crossed Netrinfl/fl mice with an EC-specific cre line (Cdh5(PAC)-creERT2)18 to generate CDH5−NTN1 mice. CDH5−NTN1 mice and their littermate controls were treated with Tamoxifen to induce EC-specific NTN1 deletion.

Evaluation of niche cell frequency in young (5-6 month old) niche specific NTN1 knockout mice revealed that while BMEC frequencies were unaltered, the frequency of BM LepR+ cells was significantly reduced in both LepR−NTN1 mice and CDH5−NTN1 mice (FIG. 1a,b). Immunofluorescence analysis of femoral sections revealed that while CDH5−NTN1 mice displayed no gross morphological alterations in their vasculature; however, LepR−NTN1 mice manifested distinct changes in vascular morphology including vessel dilation and discontinuity suggestive of impaired vascular integrity (FIG. 1c).

Analysis of vascular permeability using the modified Evans Blue Dye (EBD) extravasation assay revealed that deletion of NTN1 in BMECs did not alter vascular integrity (FIG. 1d), while NTN1 deletion in LepR+ cells increased BM vascular leakiness (FIG. 1e), indicating that BMECs require NTN1 from perivascular LepR+ cells to maintain vascular integrity within the BM. Immunofluorescence analysis also revealed that LepR−NTN1 mice and CDH5−NTN1 mice, to a lesser extent, displayed adipocyte accumulation within their BM (FIG. 1f, g). Accordingly, LepR+ MSCs isolated from LepR−NTN1 mice and CDH5−NTN1 mice demonstrated an increase in adipogenic differentiation potential (Extended Data FIG. 1d-g). Notably, impaired BM vascular integrity and adipocyte accumulation represent hallmark features of an aged BM niche and the data collectively indicates that deletion of niche-derived NTN1 induces phenotypes reminiscent of an aged BM niche19.

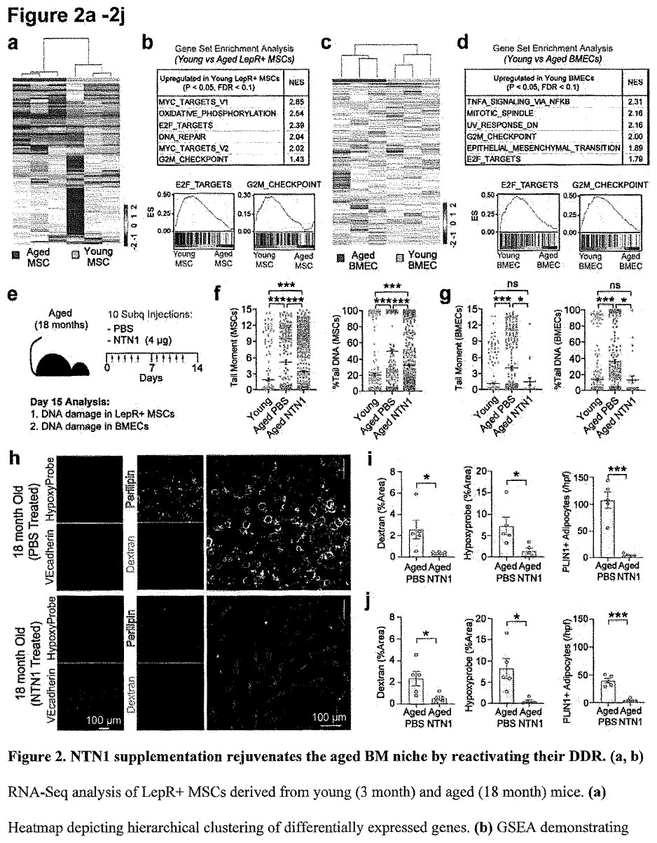

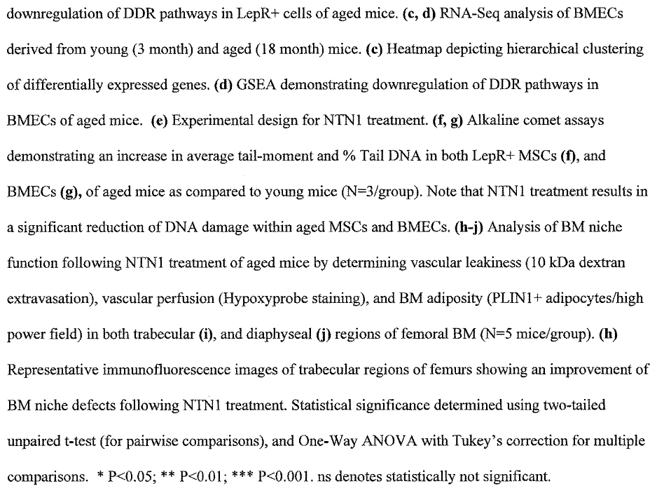

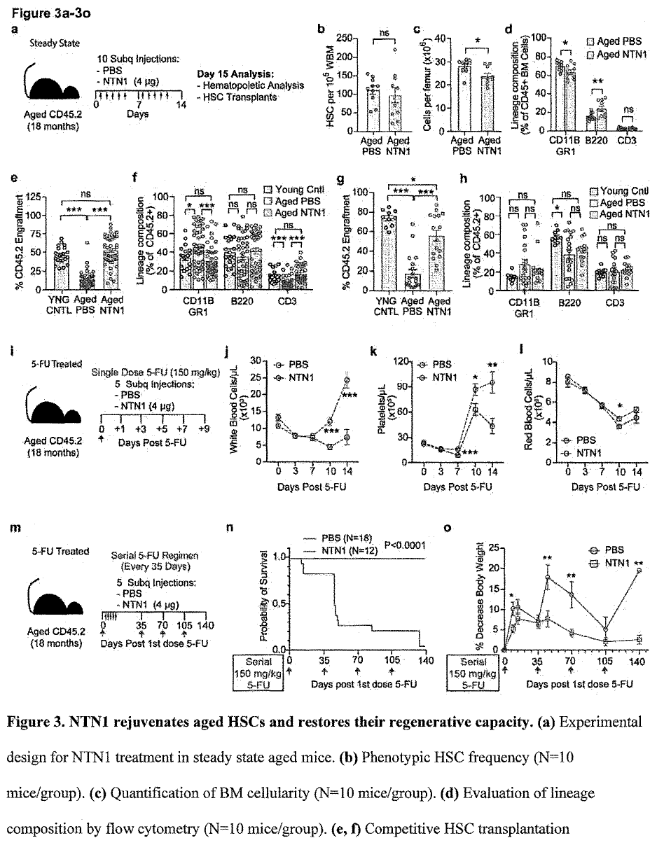

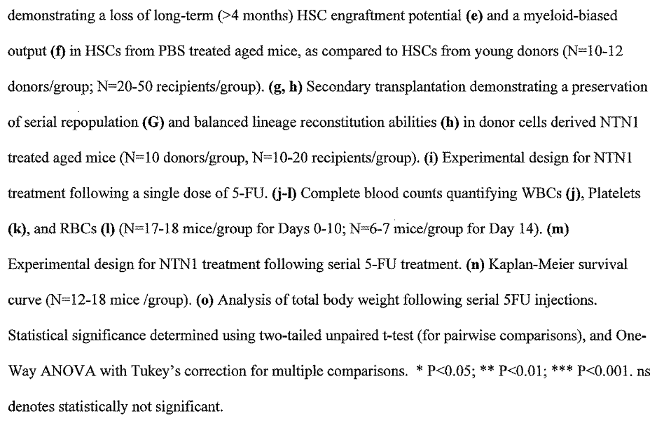

To determine whether phenotypic niche alterations observed upon deletion of niche-derived NTN1 impairs their HSC-supportive activity, analyzed were hematopoietic parameters in both LepRNTN1 mice and CDH5−NTN1 mice. While young LepR−NTN1 mice displayed normal peripheral blood lineage composition, they demonstrated a decrease in BM cellularity, and a decline in frequency of HSCs (cKIT+LineageNeg CD41NegSCA1+CD150+CD48Neg), and multipotent progenitors (MPPs; cKIT+LineageNeg SCA1+CD150NegCD48Neg), as compared to their littermate controls (Extended Data FIG. 2a-e). The decline in progenitor frequency in LepR−NTN1 mice manifested as a functional loss of progenitor activity by methylcellulose-based colony forming unit (CFU) assays (Extended Data FIG. 2f). Cell-cycle analysis revealed that HSCs from LepR−NTN1 mice had an increase in percentage of cells in the G0 phase (Extended Data FIG. 2g). Furthermore, competitive HSC transplantation assays (250 CD45.2+ donor HSCs with 106 CD45.1 WBM competitor per recipient) demonstrated that HSCs from LepR−NTN1 mice displayed an impairment in their long-term engraftment without significant changes in lineage reconstitution (Extended Data FIG. 2h-j). Young CDH5−NTN1 mice, unlike LepR−NTN1 mice, did not demonstrate changes in their BM cellularity, phenotypic HSPC frequency or progenitor activity, and displayed no alterations in their peripheral blood lineage composition (Extended Data FIG. 3a-f). However, similar to LepR−NTN1 mice, HSCs of CDH5−NTN1 mice manifested a modest increase in their quiescence (% G0). Competitive HSC transplantation assays revealed a decline in long-term HSC engraftment and a myeloid-biased reconstitution at the expense of lymphoid output, which represent hallmark characteristics of aged HSCs (Extended Data FIG. 3g-j). Collectively, these findings demonstrate that niche defects arising from niche-specific NTN1 deletion in young mice are sufficient to recapitulate HSC aging phenotypes including diminished engraftment potential, myeloid-skewed output, and an increase in HSC quiescence.

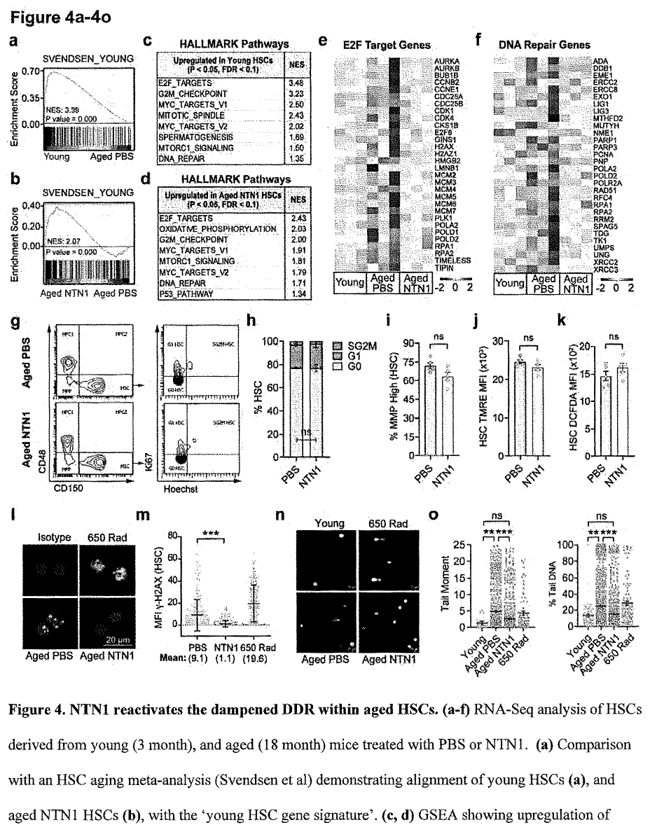

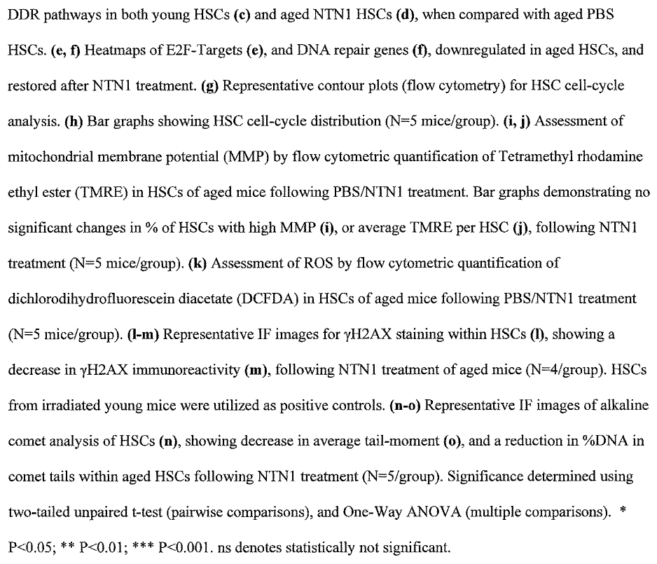

To determine whether niche-derived NTN1 is essential for preserving HSC fitness during aging, physiologically aged were LepR−NTN1 mice and CDH5−NTN1 mice for 16 months. Notably, aged LepRNTN1 mice, unlike aged CDH5−NTN1 mice, manifested marked hair greying that is typically observed following exposure to radiation20 (Extended Data FIG. 4a, b).

While HSPC frequencies were unaltered, HSCs derived from both aged LepR−NTN1 mice and CDH5−NTN1 mice manifested a significant decrease in long-term HSC engraftment along with alterations in lineage composition (Extended Data FIG. 4c-j), as compared to their respective littermate controls. Taken together, the data confirms that NTN1 derived from the bone marrow (BM) niche is essential to maintain HSC function during aging.

NTN1 Regulates DDR within the BM Miche.

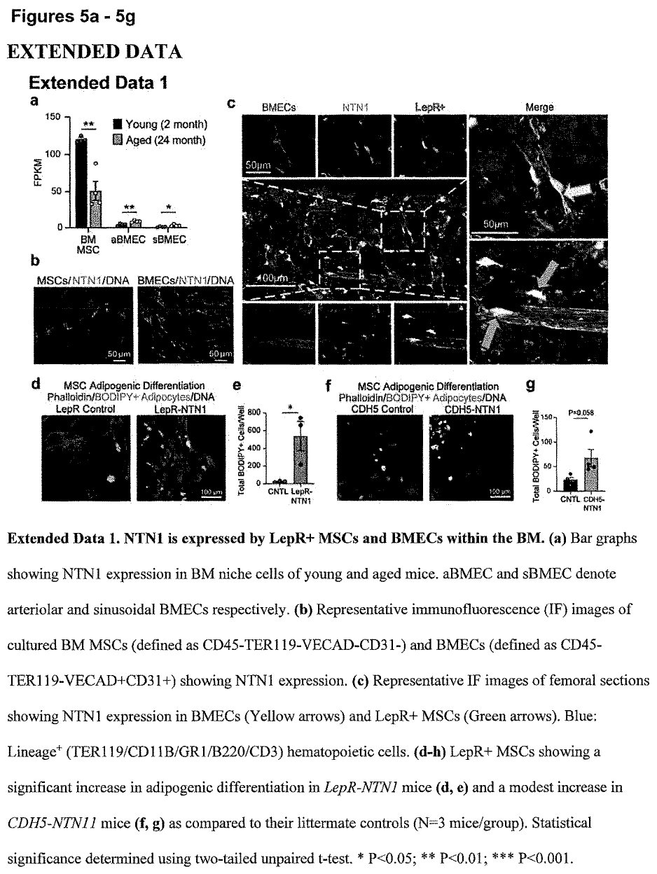

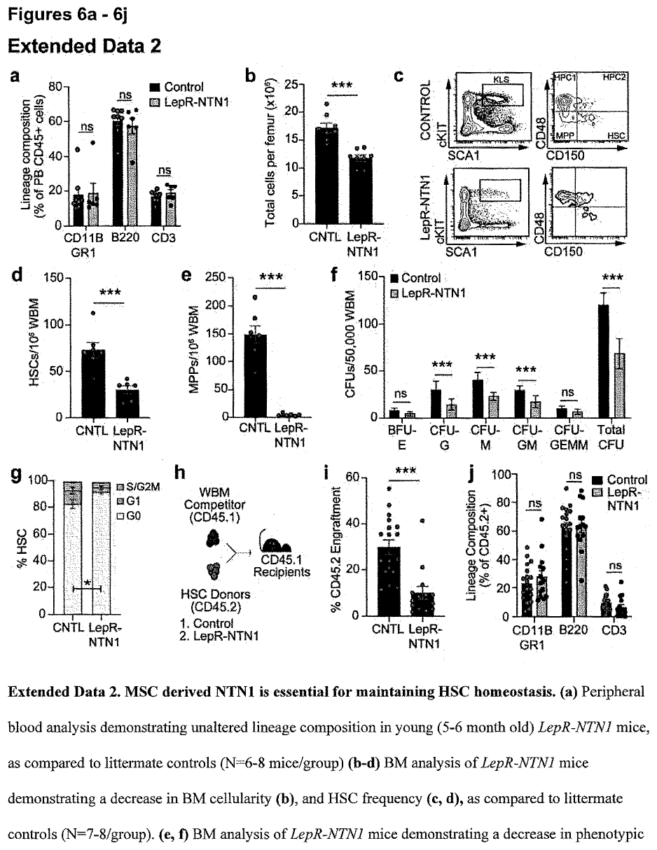

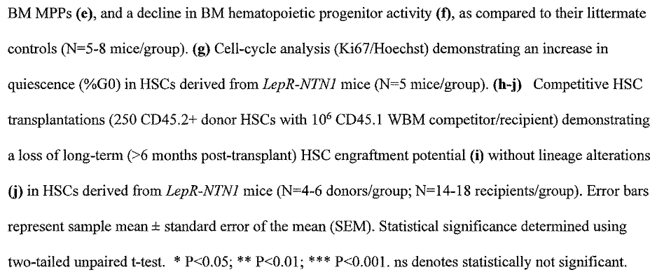

To elucidate mechanisms underlying niche defects observed upon NTN1 deletion, we performed transcriptomic analysis (RNA-Seq) on BMECs and BM LepR+ MSCs in LepR−NTN1 and CDH5−NTN1 mice (Extended Data FIGS. 5, 6 & Table S1). Gene Set Enrichment Analysis (GSEA) of the LepR+ cell transcriptomes revealed an upregulation of the ADIPOGENESIS pathway in both LepR−NTN1 and CDH5−NTN1 mice, correlating with their BM adipocyte accumulation. GSEA also revealed a striking upregulation of pathways that regulate cell-cycle and DNA damage responses (DDR) including E2F_TARGETS, G2M_CHECKPOINT, MYC_TARGETS and DNA_REPAIR pathways within LepR+ MSCs of both LepR−NTN1 and CDH5−NTN1 mice (Extended Data FIG. 5a-f & Table S2). Notably, E2F_TARGETS play crucial roles in regulation of cell-cycle and DNA Damage Responses (DDR) 21.

While cell-cycle analysis of LepR+ cells did not reveal any significant differences when compared to controls, alkaline comet assays revealed a significant increase in DNA damage in LepR+ MSCs of both LepR−NTN1 and CDH5−NTN1 mice (Extended Data FIG. 5g-j). Similar to LepR+ MSCs, GSEA of the BMEC transcriptomes also revealed an upregulation of DDR pathways and accumulation of DNA damage within BMECs, following NTN1 deletion in either LepR+ MSCs or BMECs (Extended Data FIG. 6a-j & Table S3). Taken together, the present data illustrates that loss of NTN1 in either LepR+ cells or BMECs disrupts BMEC−LepR+ niche-cell interactions within the BM, and illuminate a novel role for niche-derived NTN1 in preventing DNA damage accumulation within the BM niche.

### NTN1 Supplementation Rejuvenates the Aged BM Niche

The underlying mechanisms that drive BM niche defects during physiological aging remain largely unknown. Given that DDR disruption within niche cells of young LepR−NTN1 mice and CDH5−NTN1 mice induces premature niche aging phenotypes, we sought to determine whether aging is associated with DDR dysregulation within the BM niche. To this end, we performed transcriptomic analysis on BMECs and BM LepR+ MSCs derived from young (3 month old) and aged (18 month old) mice (FIG. 2a-d & Tables S4-S7). GSEA of both LepR+ MSC and BMEC transcriptomes revealed an over-representation of DDR pathways that were significantly downregulated during aging, raising the possibility that aging results in DNA damage accumulation within the BM niche (FIG. 2a-d & Tables S5, S7). To confirm whether aging is associated with DNA damage within the niche, and to test whether NTN1 supplementation could reverse these defects, we treated aged (18 month-old) mice with recombinant murine NTN1 over a 2 week period, and assessed the DNA damage within the BM niche (FIG. 2e-g). As indicated by the transcriptional analysis, comet assays confirmed that both LepR MSCs and BMECs derived from aged mice manifested significantly increased DNA damage, as compared to young niche cells (FIG. 2e-g). Importantly, NTN1 treatment was sufficient to ameliorate DNA damage within aged niche cells, reaffirming the role of NTN1 in regulating DDR within the niche (FIG. 2e-g).

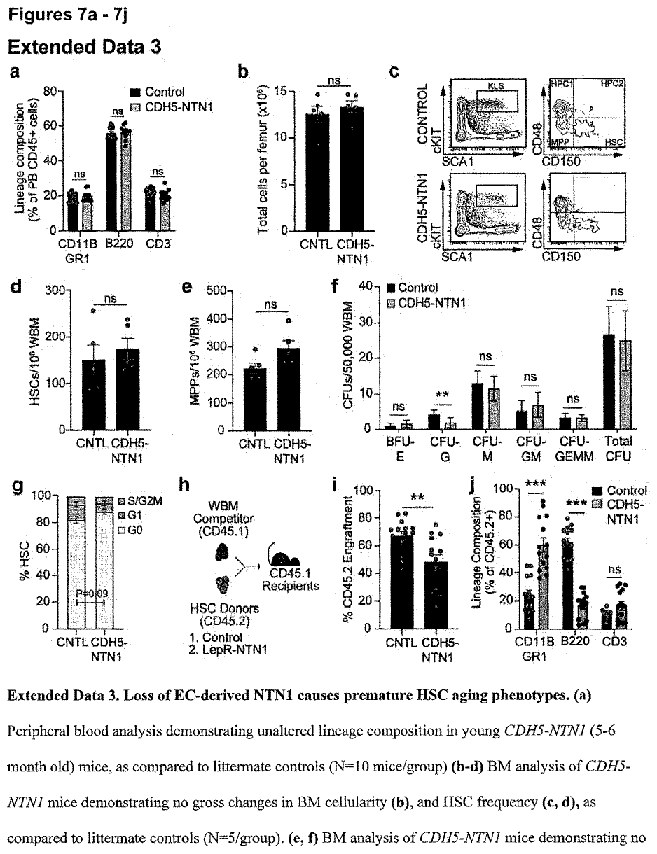

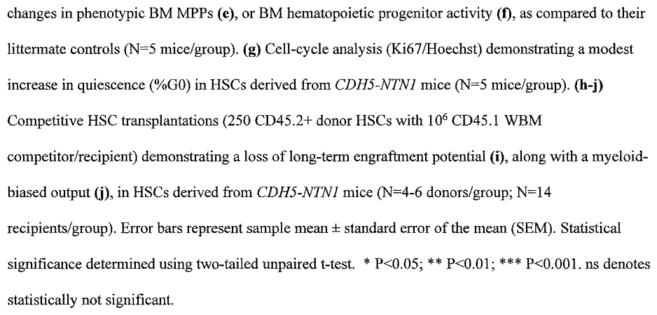

Sought next was to determine whether NTN1 mediated DNA damage resolution is sufficient to improve the functionality of an aged niche. To evaluate BM vascular leakiness and oxygenation status simultaneously, aged mice were injected with 10 kDa Dextran and Hypoxy-probe following PBS/NTN1 treatment. It was observed that treatment of aged mice with NTN1 resulted in significant reduction in their BM vascular leakiness assessed by immunofluorescence analysis of dextran extravasation (FIG. 2h-j, Extended Data FIG. 7a, b), and a marked improvement in BM perfusion evaluated by Hypoxy-probe staining. Furthermore, quantification of Perilipin+ adipocytes revealed a near complete resolution of adipocyte accumulation in the marrow of NTN1-treated aged mice. MSC differentiation assays revealed that MSCs isolated from PBS-treated aged mice manifested a near complete loss of adipogenic and osteogenic potential (Extended Data FIG. 7c, d), suggestive of a significant loss of functional LepR+ MSCs within the aged BM. Contrarily, NTN1 treatment resulted in significant improvement in adipogenic and osteogenic potential, indicating that NTN1 treatment improves LepR+ MSC activity within the aged BM (Extended Data FIG. 7c, d). Taken together, the data demonstrates that NTN1 mediated DNA damage resolution is sufficient to restore the phenotypic defects of an aged niche including restoration of BM vascular integrity and oxygenation, preservation of functional LepR+ MSCs, and a suppression of their differentiation towards adipocytes within the aged BM microenvironment.

### NTN1 Restores the Regenerative Capacity of an Aged Hematopoietic System

The resolution of phenotypic niche defects raised the possibility that NTN1 treatment rejuvenates the functionality of the aged niche in their ability maintain HSC fitness. To test this hypothesis, we performed hematopoietic analysis of aged mice treated with NTN1. While phenotypic HSC frequency remained unchanged, NTN1 treatment resulted in a modest decrease in BM cellularity, along with a reduction in myeloid cells and a concomitant improvement in B cells, as compared to PBS treated littermate controls (FIG. 3a-d). To evaluate HSC function, performed was competitive HSC transplants utilizing donor HSCs derived from aged mice treated with NTN1 or vehicle (PBS). 2500 donor HSCs (CD45.2+) were FACS purified from each donor (N=10 PBS-treated and N=10 NTN1-treated aged mice), and HSCs from each donor were competitively transplanted into 5 recipient (CD45.1+) mice (500CD45.2 + HSCs with 1.5×106 CD45.1+ WBM competitor per recipient; a total of 50 recipients for each treatment group). Simultaneously, HSCs derived from N=12 young (3 month old, CD45.2+) mice were pooled and transplanted into 20 recipients (500 CD45.2+ HSCs with 1.5×106 CD45.1+ WBM competitor per recipient), and served as young controls for comparison. As expected, HSCs from PBS-treated aged mice demonstrated the hallmark characteristics of an aged HSC including diminished long-term engraftment along with altered lineage reconstitution including increased myeloid skewing and decreased T cell reconstitution, as compared to HSCs derived from young controls (FIG. 3e,f). Remarkably, HSCs derived from NTN1-treated aged mice exhibited long-term engraftment and balanced lineage reconstitution indistinguishable from young controls (FIG. 3e, f).

To assess whether NTN1 restores self-renewal potential of aged HSCs, we performed secondary transplantation assays utilizing WBM derived from primary recipients, which revealed that hematopoietic cells derived from NTN1-treated aged mice robustly maintain their long-term engraftment (˜75% of young HSC engraftment levels) and a balanced multilineage reconstitution equivalent to young HSCs (FIG. 3g, h). Collectively, these data demonstrate that NTN1 mediated rejuvenation of the aged BM niche is sufficient to restore HSC self-renewal potential and balanced lineage reconstitution reminiscent of a young hematopoietic system.

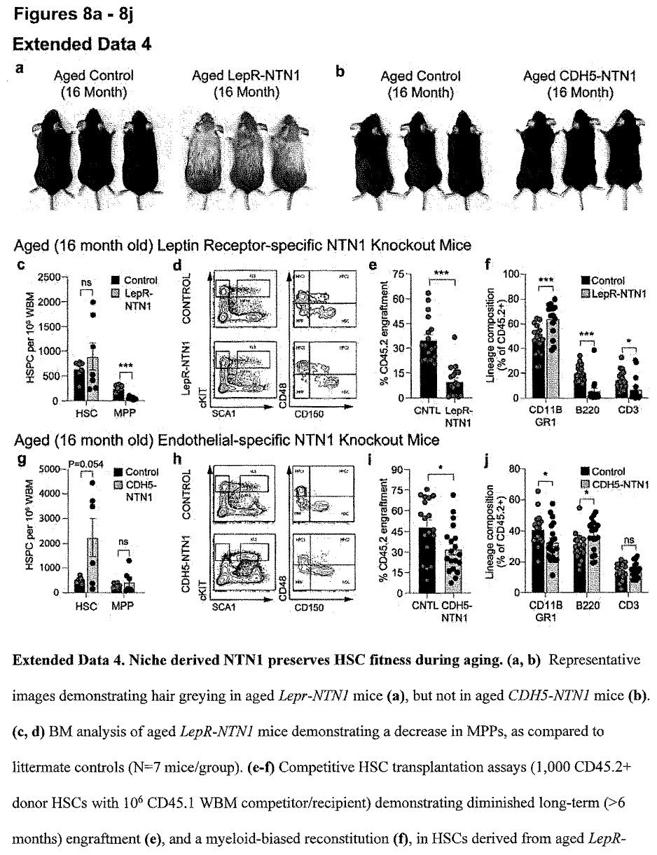

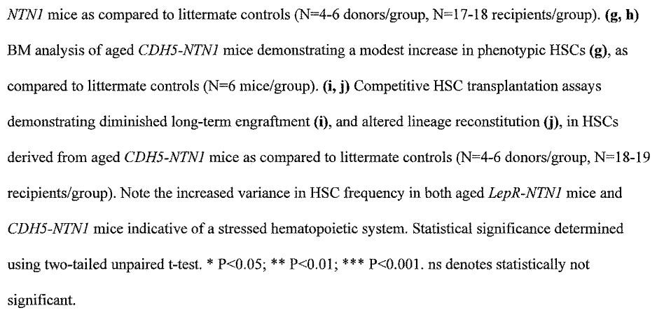

It was next sought to determine whether beneficial effects of NTN1 on the aged hematopoietic system could improve hematopoietic recovery following myelosuppressive injuries. Aged mice (18 month-old) were given a myelosuppressive dose of chemotherapy (150 mg/kg of 5 fluorouracil (5-FU)), and injected every other day with either recombinant NTN1 or PBS, for a total of 5 injections starting at Day+1 post-myelosuppression (FIG. 3i), and monitored for hematopoietic recovery (FIG. 3j-l). NTN1 treatment improved hematopoietic recovery after single dose 5-FU treatment, with platelet recovery starting at day 7 (FIG. 3l), and white and red blood cells showing significant recovery by day 10 (FIG. 3j,k). At day 10 post 5-FU, NTN1 treated aged mice demonstrated a significant recovery of their peripheral blood lymphocytes and BM cellularity, a decrease in frequency of HSCs and progenitors; as well as an overall decrease in progenitor activity, consistent with a an accelerated recovery of the hematopoietic system towards homeostatic levels (Extended Data FIG. 8a-d). To determine whether beneficial effects of NTN1 on hematopoietic regeneration extends to reducing the severe myelosuppressive stress associated with serial chemotherapy regimens, we performed serial 5-FU (150 mg/kg) treatments on aged mice every 35 days for a total of four treatments, and assessed their survival for 140 days (FIG. 3mo).

Following the first injection of 5-FU, aged mice were treated with either PBS or NTN1 every other day, for a total of 5 injections (FIG. 3m), and mice did not receive any NTN1 treatment following subsequent doses of 5-FU. In line with the known loss of HSC self-renewal ability and regenerative capacity during aging, ˜75% of the PBS treated aged mice succumbed to hematopoietic failure following the first two doses of 5-FU, with only 1 out 18 mice surviving all four doses (FIG. 3n).

Conversely, NTN 1 treated aged mice demonstrated 100% survival (12/12) (FIG. 3n). Additionally, serial 5-FU treatment resulted in significant loss of body weight in surviving PBS treated aged mice following every injection of 5-FU, while aged mice treated with NTN1 demonstrated a better preservation of their body weight (<10% average weight loss) throughout the duration of serial 5-FU regimen (FIG. 3o). These data indicate that NTN1 treatment can enhance self-renewal and regenerative capacity of aged HSCs, as well as protect the hematopoietic system from progressive loss of stem cell reserves and preserve body weight; toxicities commonly associated with chemotherapy in the elderly.

### NTN1 Reactivates the Dampened DNA Damage Response Within Aged HSCs

To elucidate mechanisms underlying NTN1-mediated HSC rejuvenation, we performed RNASeq analysis on HSCs derived from aged mice treated with PBS or NTN1 (herein referred to as aged-PBS HSCs or aged-NTN1 HSCs, respectively), as well as HSCs derived from young mice (referred to as young HSCs) (FIG. 4 & Tables S8, S9). To determine whether NTN1 restores transcriptional alterations within aged HSCs, we compared the transcriptomes of aged-NTN1 HSCs with a meta-analysis of 10 previously published HSC aging transcriptomic datasets22. GSEA revealed that aged-NTN1 HSCs, similar to young HSCs, aligned with the ‘young HSC signature’, indicating that treatment with NTN1 is sufficient to restore the principal age-deregulated transcriptional networks within HSCs (FIG. 4a, b). To identify molecular pathways modulated by NTN1 that promote HSC rejuvenation, analyzed were pathways that were differentially expressed during aging (young versus aged-PBS HSCs) as well following NTN1 infusion (aged-PBS versus aged-NTN1 HSCs). GSEA revealed a marked overlap of pathways upregulated in young HSCs and aged-NTN 1 HSCs as compared to aged-PBS HSCs (FIG. 4c,d & Table S9). As observed in aged BM niche cells, there was an over-representation of DDR related pathways including E2F_TARGETS, G2M_CHECKPOINT, MYC_TARGETS and DNA_REPAIR that were upregulated in young HSCs and aged-NTN1 HSCs as compared to aged-PBS HSCs (FIG. 4c,d & Table S9). Evaluation of E2F_TARGETS and DNA_REPAIR pathways demonstrated downregulation of key DDR genes within aged-PBS (FIG. 4e,f). Cell-cycle analysis demonstrated that NTN1 treatment did not result in gross changes to the G0/G1/SG2M fractions in HSCs of aged mice (FIG. 4g-h).

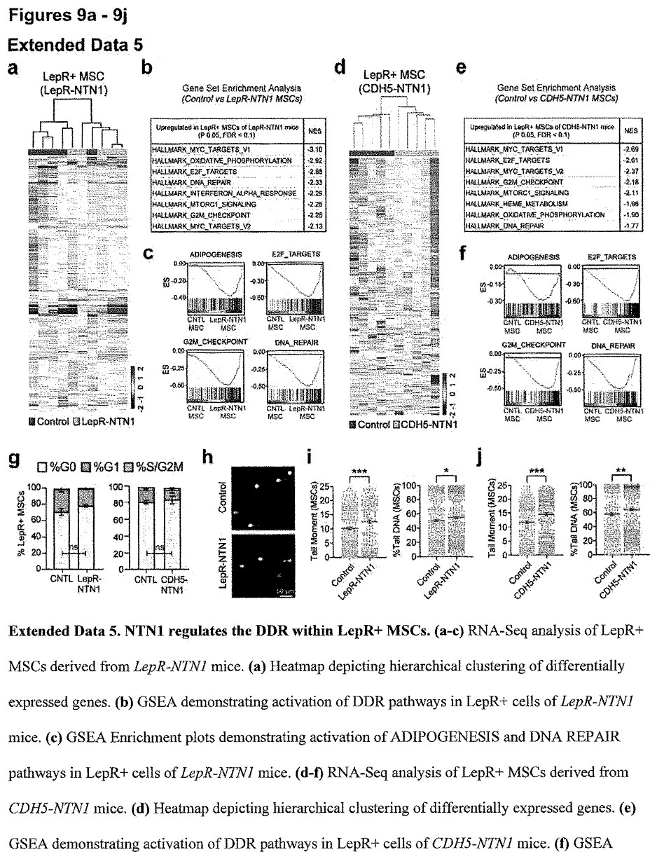

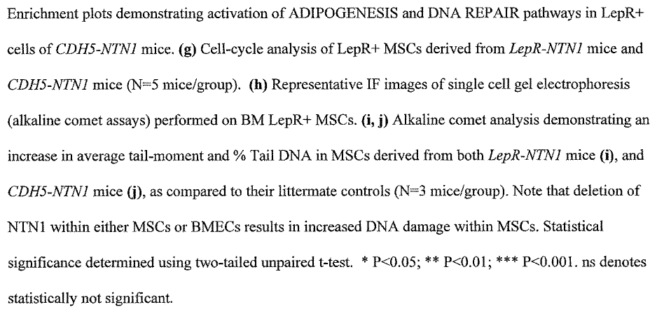

Besides cell-cycle and DDR pathways, aged-NTN1 HSCs also demonstrated an upregulation of the OXIDATIVE_PHOSPHORYLATION pathway recently shown to regulate HSC function during aging23 (FIG. 4d). However, NTN1 treatment did not alter mitochondrial membrane potential or reactive oxygen species (ROS) levels within HSCs of aged mice (FIG. 4i-k), indicating that NTN1 mediated aged HSC rejuvenation likely results from reactivation of dampened DDR within aged HSCs24,25. Supporting this, GSEA utilizing REACTOME and KEGG databases (Table S9) confirmed that aging is associated with a global downregulation of DNA repair genes and pathways in HSCs including Base Excision Repair (BER), Nucleotide Excision Repair (NER), Homologous Recombination (HR) and Mismatch Repair (MMR) (Extended Data FIG. 9a, b).

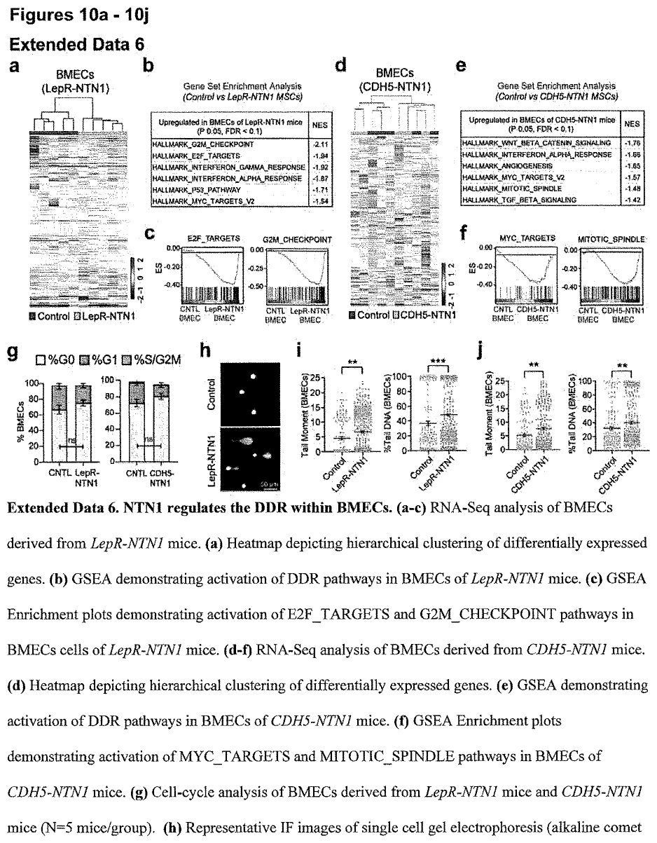

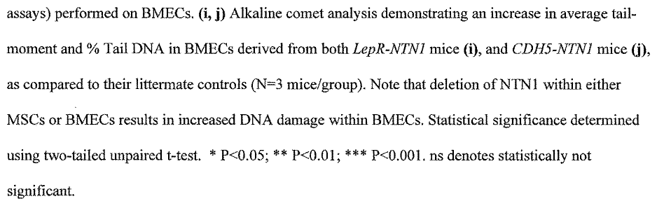

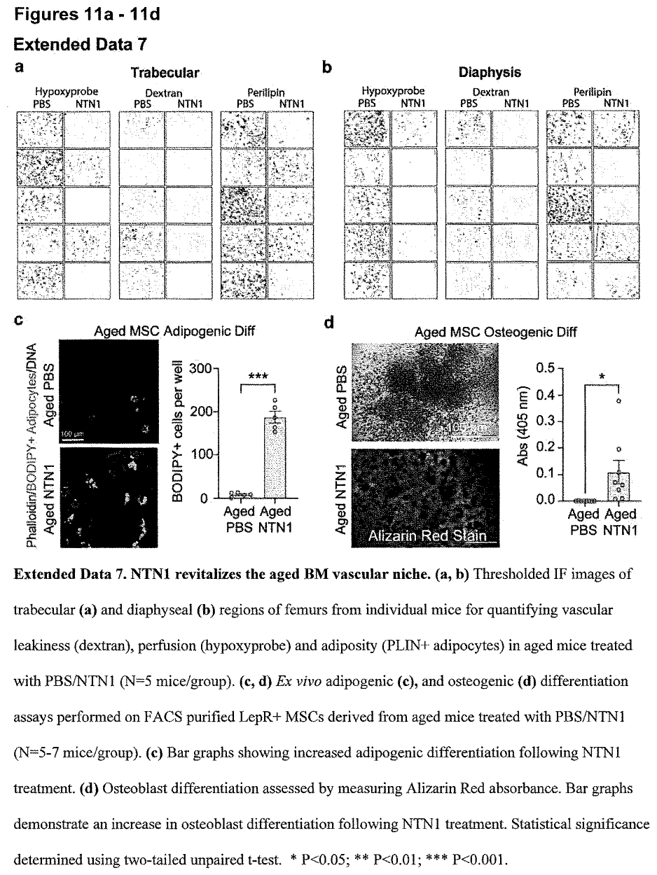

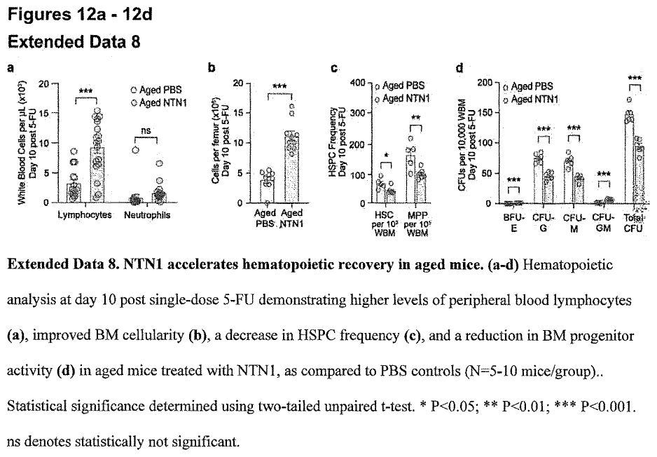

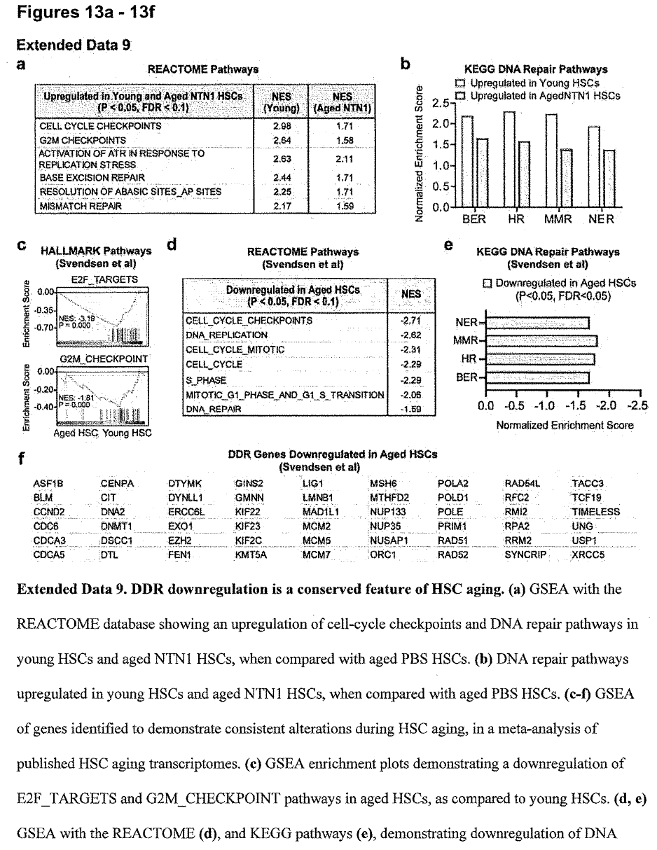

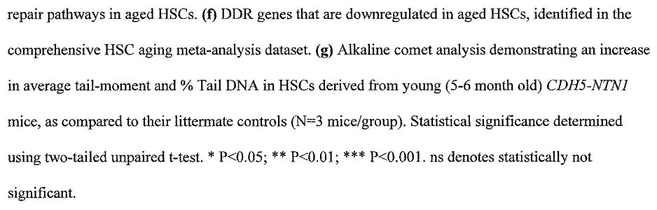

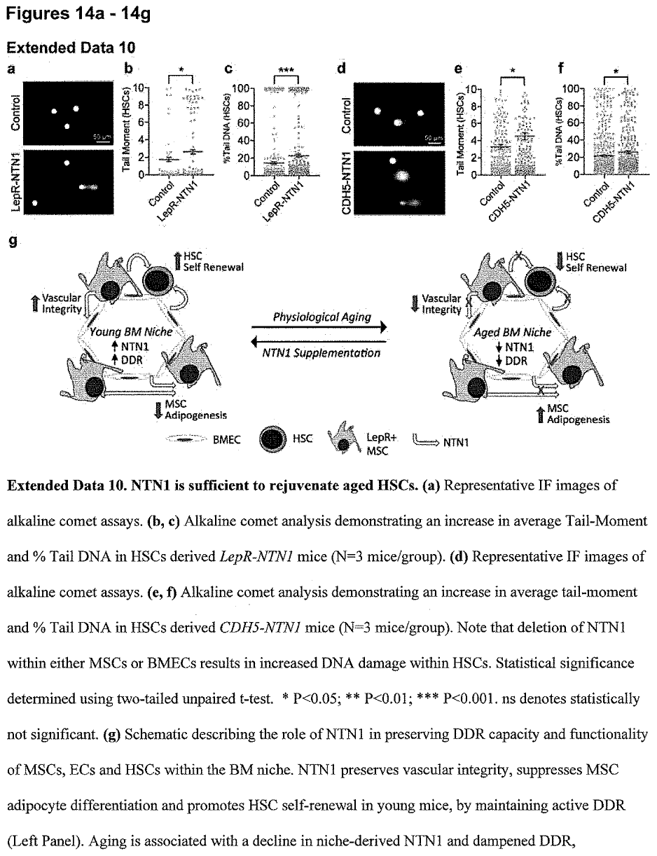

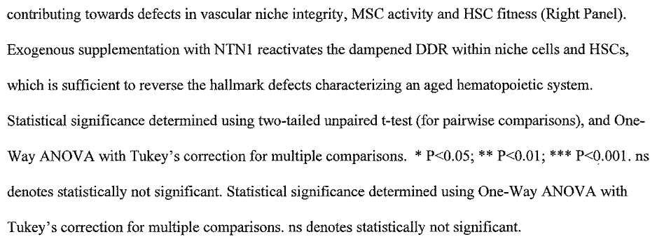

Notably, treatment with NTN1 restored the expression of DDR genes and pathways within aged HSCs. To ascertain whether DDR downregulation is a conserved feature of HSC aging, performed was GSEA of genes dysregulated within aged HSCs identified in the meta-analysis of published HSC aging transcriptomes (Table S10)22. GSEA revealed that downregulation of DDR pathways and genes represents a consistent feature of HSC aging (Extended Data FIG. 9c-f) that explains the accumulation of DNA strand breaks and stalled replication forks within aged HSCs26,27. To verify whether NTN1 mediation DDR activation is sufficient to resolve DNA damage within aged HSCs, we quantified the levels of γ-H2AX which revealed that NTN1 treatment resulted in a reduction in their nuclear γ-H2AX foci to basal levels (FIG. 4l, m). Alkaline comet assays confirmed that NTN1-treatment resolves DNA damage in aged HSCs to levels observed in young mice (FIG. 4o), demonstrating that NTN1 supplementation reactivates DDR and resolves DNA damage within aged HSCs. Notably, HSCs derived from LepR−NTN1 and CDH5−NTN1 mice displayed increased DNA damage, confirming that niche-derived NTN1 directly regulates DDR within HSCs (Extended Data FIG. 10a-f). Taken together, these data demonstrate that a young niche provides adequate NTN1 to support niche integrity and HSC self-renewal by maintaining a robust DDR (Extended Data FIG. 10g). Age-related decline in niche-derived NTN1 contributes towards a dysfunctional niche and damaged HSCs resulting from dampened DDR and DNA damage accumulation. Supplementation of aged mice with NTN1 resolves DNA damage within the BM niche and HSCs, and rejuvenates an aging hematopoietic system.

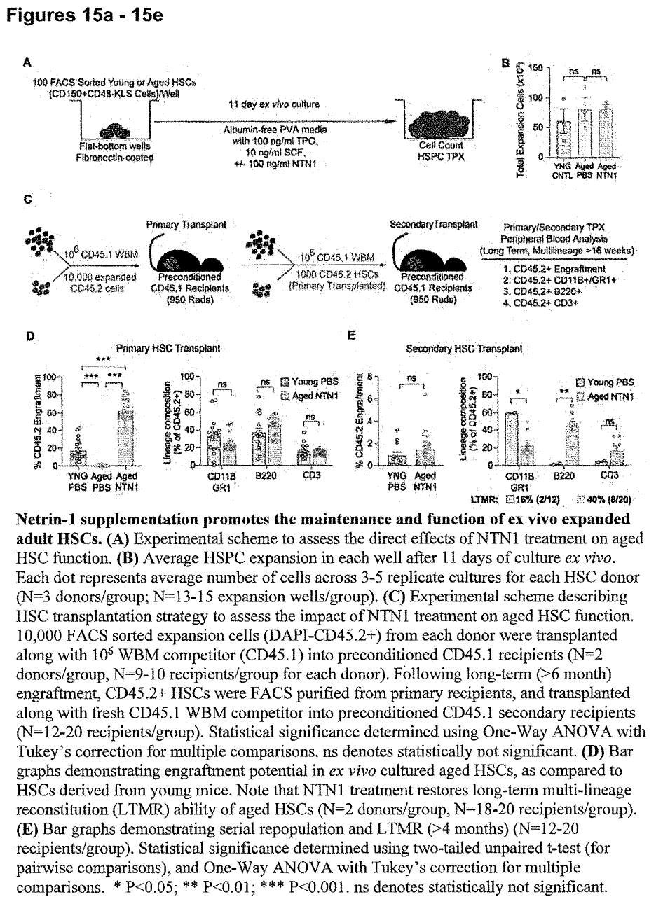

The present investigator herein demonstrates that the rejuvenating properties of Netrin-1 (NTN1) treatment on aged hematopoietic stem cells (HSCs) arise from its beneficial effects on the aging microenvironment, a direct effect on the HSC itself, or a combination thereof. To determine whether direct effects of NTN1 on aged HSCs are sufficient for therapeutic rejuvenation, the present investigator took advantage of a recently described ex vivo HSC expansion platform utilizing polyvinyl alcohol (PVA) in a serum-free medium with low dose KitL (10 ng/ml) and TPO (100 ng/ml) that expands transplantable HSCs ex vivo, allowing for interrogation of novel compounds with putative HSC regulatory effects. Using the PVA expansion protocol, we cultured HSCs derived from aged mice (18 month-old) for 11 days, in the presence of recombinant NTN1 or vehicle (PBS) (FIG. 15A). Additionally, HSCs derived from young mice (3 month-old) cultured in PVA medium with vehicle (PBS) served as controls for comparison.

While the total number of expanded cells (FIG. 15B) following 11 days of ex vivo culture did not reveal significant differences across all 3 groups, competitive transplantation (FIG. 15C) of equal numbers of expansion cells (10,000 FACS sorted CD45.2+ expansion cells with 106 CD45.1 WBM) competitor per recipient) into preconditioned CD45.1 recipients (N=18-20 recipients/group) showed stark differences in their long-term (>6 month) engraftment ability. While expansion cells derived from PBS-treated aged HSCs manifested a complete loss of long-term engraftment potential, aged HSCs cultured in the presence of NTN1 demonstrated robust long-term engraftment and balanced multi-lineage reconstitution, outperforming expansion cells derived from young HSCs (FIG. 15D). To test the self-renewal ability of expanded HSCs, we performed secondary transplantation assays (FIG. 15E) utilizing FACS sorted HSCs from primary donors (CD45.2+), and transplanting them into secondary recipients (CD45.1+), along with freshly isolated WBM competitor cells (1000 CD45.2+ HSC plus 106 CD45.1 WBM competitor per recipient). While HSCs derived from both young control and NTN1 treated aged primary donors displayed similar levels of long-term (>4 months) engraftment, HSCs derived from NTN1 treated aged mice had more recipients with long term multilineage reconstitution (LTMR), while maintaining balanced lineage reconstitution when compared to young controls (FIG. 4I). Taken together, these data suggest that direct effects of NTN1 on aged HSCs are sufficient to rejuvenate their self-renewal potential.

### Aging and NTN1 Signaling Regulates Leukemic Progression and Survival.

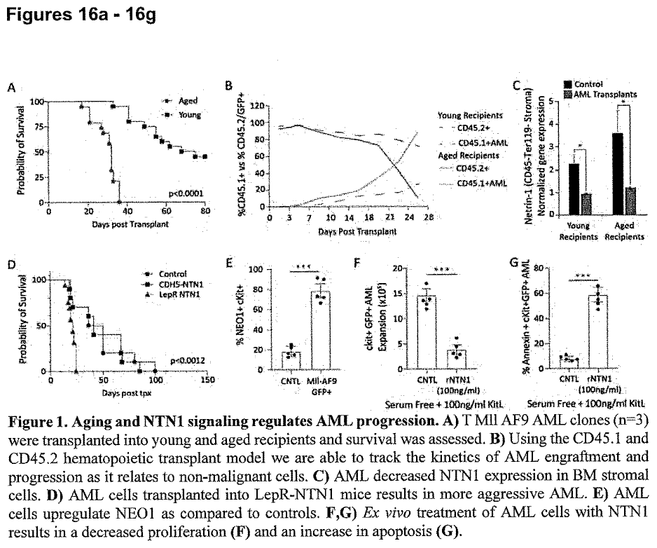

The risk of acute myeloid leukemia (AML) increases with age and accumulating evidence indicates that the microenvironment provides the extrinsic signals necessary for the progression of AML. Therefore, we wanted to determine if an aged microenvironment is more conducive to the rapid progression of AML. To this end, we transplanted 2×105 cKit+ AML cells into non-irradiated young (3 months) and aged (18 months) mice (FIG. 16A). We found that aged mice succumbed to AML significantly faster than young controls (FIG. 16A). To track the kinetics of AML engraftment and progression while simultaneously tracking endogenous, non-malignant hematopoietic cells, we generated M11-AF9 AML clones utilizing CD45.1 mice. Transplanted then were CD45.1+cKit+GFP+AML cells into non-irradiated CD45.2 young or aged mice and tracked the progression of disease versus normal hematopoietic cells.

It was found that in aged recipients, as early as 21 days post-transplant of AML cells the percentage of AML cells in the peripheral blood was beginning to surpass the percentage of non-malignant cells. Conversely, in young recipients, at day 21 post-transplant, the percentage of AML cells averaged 10-15% (FIG. 16B). Because the present investigator hypothesized that the NTN1 signaling axis preserves the vascular and hematopoietic systems during aging, we next set out to determine the importance of BM niche-derived NTN1 for the progression of AML. Analysis of NTN1 expression levels in total BM stromal cells revealed that NTN1 expression was downregulated in both young and aged AML recipient mice (FIG. 16C). Moreover, it was found by the present investigator herein that transplantation of AML into non-irradiated LepR−NTN1 mice resulted in a significant decrease in survival of these mice as compared to control and CDH5−NTN1 mice (FIG. 16D), indicating that LepR-derived NTN1 negatively affect AML aggressiveness.

Interestingly, when screened 5 independent clones of MII-AF 9 AML cells, we found that NEO1 expression was increased (FIG. 16E). Finally, because we hypothesize a protective effect of NTN1 on aging, we wanted to determine if exogenous NTN1 would promote or block the expansion of AML cells ex vivo. We found that the addition of NTN1 to cultures of AML resulted in a decrease in overall expansion (FIG. 16F) with a corresponding increase in apoptosis (FIG. 16G). Taken together, these data suggest that NTN1 is a novel therapeutic to treat AML with two modes of action: preserving hematopoietic/vascular function while simultaneously targeting AML cells.

### Discussion

An unresolved issue regarding aging is whether regenerative potential of aged HSCs and their supportive niches can be restored to youthful levels. It was recently shown that aged HSCs are refractory to systemic anti-aging interventions that have shown improvements in other stem cell systems28. Contrarily, few studies have demonstrated that some defects of an aged HSC can be mitigated by targeting HSC-intrinsic mechanisms23,29-32 or their supportive niche cells19,33-36, evident by improvements in HSC engraftment potential and reductions in their myeloid skewing. However, whether age-associated decline of HSC self-renewal ability, the defining property underlying their regenerative potential, can be functionally restored remained unknown.

Here, the present investigator demonstrated that NTN1 treatment of aged mice rejuvenates their BM niche and restores HSC regenerative potential. The beneficial effects of NTN1 on the aged hematopoietic system translate into a strong survival benefit and preservation of body weight during serial myelosuppressive chemotherapy. These findings have therapeutic implications for improving hematopoietic health span in the elderly and improving outcomes following chemotherapeutic regimens.

While significant progress has been made in unraveling the role of BM niche derived extrinsic signals in regulating HSC function, the signaling mechanisms between cellular constituents comprising the niche that regulate vascular integrity, oxygenation, and adiposity within the aging BM remain poorly understood. Here, it was identified that NTN1 is a linchpin molecule that regulates BMEC-LepR+ MSC niche interactions reciprocally by preserving the integrity of the BM vascular niche and preventing adipocyte accumulation.

Periarteriolar smooth muscle cells (SMCs) were initially described as a putative niche cell that provides NTN1 to HSCs within the BM13. However, conditional deletion of NTN1 within SMCs did not recapitulate HSC defects observed upon ubiquitous deletion of NTN113. Here, we define LepR+ MSCs and BMECs as the bona fide sources of niche-derived NTN1 that not only maintain HSC fitness, but also preserve niche integrity within the BM. Demonstrated herein is that niche-derived NTN1 plays an essential role in maintaining active DDR within MSCs and BMECs.

The present investigator identified that aging is associated with a downregulation of DDR within both BMECs and MSCs, identifying a conserved mechanism underlying age-related defects within the BM niche. Notably, diminished DDR within aged niche cells likely explains the higher chemo-sensitivity observed during aging.

Importantly, the findings demonstrate that niche-derived NTN1 is essential to prevent DNA damage within HSCs, illuminating a novel role for BM niche cells in regulating HSC DDR. By performing transcriptomic analysis of aged HSCs, we confirm that DDR downregulation is a conserved attribute of HSC aging as described previously24,25,37 and our findings indicate that age-related decline in niche-derived NTN1 likely underlie DDR downregulation within aged HSCs. Observations from human genetic disorders and studies employing diverse array of genetic model systems have unequivocally established that DDR insufficiency causes premature aging phenotypes and that aging results in generalized impairment of DDR capacity2,38-40.

However, what remained unresolved was whether DDR reactivation is sufficient to rejuvenate stem cell regenerative potential during aging, particularly within the mammalian system2,38,39.

Indeed, accumulation of DNA damage is a hallmark feature of aging and widely considered the central cause of the aging process41. However, identification of master regulators of DNA repair that affects multiple DDR pathways and development of strategies to reactivate the DDR in aging tissues is lacking and is currently a major focus in aging research2,38,41.

Here, it was demonstrated that NTN1 mediated reactivation of dampened DDR is sufficient to rejuvenate an aged BM niche and restore HSC function to youthful levels. The findings raise the possibility that DDR downregulation could underlie a fundamentally conserved attribute of tissue specific stem cells and their niches. Supporting this, a recent study comparing the transcriptomes of young and aged muscle stem cells (MuSCs) identified that E2F_TARGETS and G2M_CHECKPOINT were amongst the most significantly downregulated pathways during aging42.

Importantly, the same pathways were upregulated following exercise in rejuvenated aged MuSCs42. While activities of specific DNA repair pathways in aged HSCs and niche cells were not explored owing to the limitations associated with rare cell populations, NTN1 mediated upregulation of diverse DDR pathways at the transcriptional level, coupled with resolution of DNA damage in aged niche cells and HSCs are indicative of global upregulation of DDR pathways.

Although the effects of NTN1 infusion on other systemic aging parameters were not investigated in this study, the beneficial effects of NTN1 on diverse cell types including BM MSCs, ECs and HSCs, along with its ability to preserve body weight during serial chemotherapy, are suggestive of therapeutic potential beyond the BM. Since DDR plays a critical role during diverse array of pathophysiological conditions including clonal disorders, regeneration following myelosuppressive therapies, neurodegenerative disorders, and the overall systemic aging process43, it is imperative to understand the receptors/signal transducers/effectors in HSCs and niche cells that are involved in NTN1 mediated activation of the DDR. Provided below and herein are methods for that understanding.

### Methods

### Animals

All murine experiments were conducted in accordance with the Association for Assessment and Accreditation of Laboratory Animal Care, Intl. (AAALAC) and National Institutes of Health (NIH) Office of Laboratory Animal Welfare (OLAW) guidelines, and under the approval of the Hackensack Meridian Health and Center for Discovery and Innovation Institutional Animal Care and Use Committee (IACUC). Young and aged C57BL/6 (CD45.2, JAX Stock No: 000664) mice were purchased from the National Institutes on Aging, and the Jackson Laboratory (Bar Harbor, ME). B6.129(Cg)-Leprtm2(cre)Rck/J (JAX Stock No. 008320) mice, B6.129(SJL)-NTN1tm1.1Tek/J (JAX Stock No. 028038) mice and B6.SJLPtprca Pepcb/BoyJ (CD45.1; JAX stock No. 002014) mice were purchased from the Jackson Laboratory. Cdh5(PAC)-creERT2 mice were obtained from Ralf H. Adams at The Max Planck Institute for Molecular Biomedicine 44. Mice were acclimatized in the vivarium for at least 2 weeks prior to experimental use. Cdh5(PAC)-creERT2, B6.129(Cg)-Leprtm2(cre)Rck/J and B6.129(SJL)-NTN1tm1.1Tek/J mice were maintained on a C57BL/6J (CD45.2) genetic background. All mice were housed in NexGen Individually Ventilated Cages (IVC) with HEPA-filtered air exchange (Allentown, Inc.) and maintained on PicoLab Rodent Diet 20 (Lab Diet 5053) and water ad libitum.

To induce Cdh5(PAC)-creERT2-mediated recombination, mice were maintained on Custom Teklad 2020 Feed supplemented with 0.025% w/w tamoxifen (Envigo) ad libitum starting at 6-10 weeks of age for four consecutive weeks. Age matched cre-negative littermate mice underwent the same tamoxifen induction regimen, and were utilized as controls. Mice were allowed to recover for at least 4 weeks post-tamoxifen induction prior to experimental analysis. All mice were maintained in specific-pathogen-free housing.

### NTN1 Treatment

Recombinant murine NTN1 protein (R&D Systems 1109-N1/CF) was reconstituted in PBS at 100 μg/mL and stored at −20° C. in single-use aliquots. 4 μg NTN1 was diluted to a final volume of 100 μL in PBS, and injected subcutaneously. PBS injections served as vehicle controls.

### 5-FU Treatment

5-Fluorouracil (Fresenius Kabi 101710) was diluted in PBS to achieve a dose of 150 mg/kg body weight, and administered intraperitoneally in a volume of 150 μL.

### Buffers and Media

1. Phosphate Buffered Saline (PBS pH 7.4, Corning 21-040 CV).
2. Magnetic Activated Cell Sorting (MACS) buffer: PBS without Ca++/Mg++ (pH 7.4) (Corning 21-040-CV) containing 0.5% bovine serum albumin (BSA; Fisher Scientific BP1605) and 2 mM EDTA (Corning 46-034-CI).
3. Digestion buffer: Hanks Balanced Salt Solution (Life Technologies 14065) containing 20 mM HEPES (Corning 25-060-CI), 2.5 mg/mL Collagenase A (Roche 11088793001), and 1 unit/mL Dispase II (Roche 04942078001).
4. Endothelial Growth Medium. 1:1 ratio of Low-glucose DMEM (Thermo Fisher Scientific 11885-084) and Ham's F-12 (Corning 10-080-CV), supplemented with 20% heat-inactivated FBS (Denville Scientific FB 5002-H), 1% antibiotic-antimycotic (Corning 30-004-CI), 1% non-essential amino acids (Corning 25-025-CI), 10 mM HEPES (Corning 25-060-CI), 100 μg/mL heparin (Sigma-Aldrich H 3149), and 50 μg/mL endothelial cell growth supplement (Corning 356006).

### BMEC and MSC Cultures for NTN1 Immunohistochemistry

BMEC and MSC cultures were established as previously described 45. Briefly, femurs and tibiae were gently crushed using a mortar and pestle, enzymatically dissociated with Digestion buffer for 15 minutes at 37° C., filtered (40 μm; Corning 352340), and washed in MACS buffer. WBM was depleted of terminally differentiated hematopoietic cells using a murine Lineage Cell Depletion Kit (Miltenyi Biotech 130-090-858) according to the manufacturer's recommendations. BMECs were immunopurified from cell suspensions using sheep anti-rat IgG Dynabeads (Thermo Fisher Scientific 11035) pre-captured with a CD31 antibody (MEC13.3; Biolegend) in MACS buffer, according to the manufacturer's suggestions. CD31-enriched BMECs, and CD31-depleted stromal cells were cultured in fibronectin-coated tissue culture plates in endothelial growth media and were transduced with 104 pg myrAkt1 lentivirus per 3×104 cells/cm2.

Akt-transduced BMECs and stromal cells were selected for seven days in serum- and cytokine-free StemSpan SFEM (StemCell Technologies, Inc. 09650) media. BMEC and stromal cells were stained with antibodies against VECAD (BV13; Biolegend), CD31 (390; Biolegend), and CD45 (30-F11; Biolegend) and FACS sorted (FACS ARIA III, BD Biosciences) for purity.

BMECs were defined as CD45−CD31+ VEcadherin+, and stromal cells were defined as CD45−CD31− VEcadherin−. Cells were cultured in endothelial growth medium at 37° C., 5% CO2, and 20% O2in 70 % relative humidity. Growth media was replaced every two days and cells were passaged 1:2 at 95% confluency with Accutase Cell Detachment Solution (Biolegend 423201) according to the manufacturer's suggestions.

### Hematopoietic, BMEC and BM Stromal Cell Quantification

To quantify total hematopoietic cells, femurs were gently crushed with a mortar and pestle, enzymatically disassociated with Digestion buffer for 15 minutes at 37° C., filtered, and washed in MACS buffer. Viable cell numbers were quantified using a hemocytometer with Trypan Blue (Life Technologies 15-250-061) dye exclusion. To quantify hematopoietic stem and progenitor cells (HSPCs) in the BM, femurs and tibiae were flushed using a 26 G×½ needle with MACS buffer. To quantify BMECs, LEPR+ cells, and osteoblasts, femurs were gently crushed with a mortar and pestle, enzymatically disassociated with Digestion buffer for 15 minutes at 37° C., and filtered and washed in MACS buffer. Cells were surface stained using fluorochrome-conjugated antibodies as per manufacturer recommendations, and analyzed by flow cytometry. Gating strategy described under flow cytometry.

### Hematopoietic Progenitor Activity

Colony-forming units (CFUs) in semi-solid methylcellulose were quantified to assess hematopoietic progenitor activity. WBM was flushed from femurs and tibiae using a 26 G×½ needle with MACS buffer. Viable cell counts were determined with a hemocytometer using Trypan Blue. WBM cells were plated in duplicate in Methocult GF M3434 methylcellulose (StemCell Technologies 03444) according to the manufacturer's recommendations. Colonies were scored for phenotypic CFU-GEMM, CFU-GM, CFU-G, CFU-M, and BFU-E colonies using a SMZ1270 Stereo-Microscope (Nikon).

### Flow Cytometry

Prior to cell surface staining, Fc receptors were blocked using an antibody against CD16/32 (93; Biolegend) in MACS buffer for 10 minutes at 4° C. For CMP/GMP/MEP staining, samples were blocked with 10% normal rat serum for 10 minutes at 4° C. Blocked samples were subsequently stained with fluorochrome-conjugated antibodies in MACS buffer for 30 minutes at 4° C. Samples stained with biotinylated anti-LEPR antibody were washed and stained with Streptavidin-conjugated fluorochromes for 30 minutes at 4° C. Stained cells were washed in MACS buffer and fixed in 1% paraformaldehyde (PFA, MP Bio 0219998380) in PBS with 2 mM EDTA (Corning 46-034-CI). Sample data was collected and analyzed using a flow cytometer (Fortessa, BD Biosciences) with FACS DIVA 8.0.1 software (BD Biosciences). Fluorescence compensation was performed utilizing single-stained controls of BM cells. Gates were established using unstained controls and standard fluorescence minus one strategies. List of antibody clones utilized for Flow Cytometry are included in Table S11. Gating strategy for flow cytometry were described previously 46, and defined as follows:
KLS: Lineage (TER119/CD11B/GR1/B220/CD3)−cKIT+SCA1+
HSC: Lineage—cKIT+SCA1+CD48−CD150+
MPP: Lineage—cKIT+SCA1+CD48−CD150−
HPC-1: Lineage—cKIT+SCA1+CD48+CD150−
HPC-2: Lineage—cKIT+SCA1+CD48+CD150+
CLP: Lineage—cKITlowSCA1lowFLT3+IL7Rα+
CMP: Lineage—cKIT+SCA1—CD34+CD16/32−
GMP: Lineage—cKIT+SCA1—CD34+CD16/32+
MEP: Lineage—cKIT+SCA1—CD34—CD16/32−
Pre Pro B: sIgM—B220+CD43+CD24−
Pro B: sIgM—B220+CD43+CD24+
Pre B: sIgM—B220+CD43−CD24+
Myeloid: CD45+CD11B+GR1+
B cells: CD45+B220+
T cells: CD45+CD3+
BMECs: CD45−TER119−CD31+VEcadherin+
BM LEPR+cells: CD45−TER119−CD31−LEPR+
BM Osteoblasts: CD45−TER119−CD31−SCA1−CD51+

### Peripheral Blood Analysis

Peripheral blood (PB) was collected using 75 mm heparinized glass capillary tubes (Kimble-Chase 41B2501) via retro-orbital sinus bleeds into micro-centrifuge tubes containing PBS with 10 mM EDTA. Blood indices were analyzed using an automated hematology analyzer (Element HT5, Heska). To quantify steady state lineage+hematopoietic cells and multi-lineage HSC engraftment, PB was depleted of red blood cells (RBC Lysis Buffer; Biolegend 420301) according to the manufacturer's recommendations, stained with indicated fluorophore-conjugated antibodies, and analyzed using flow cytometry.

### Competitive Transplantation

Adult CD45.1 transplant recipient mice (12-16 week old) were pre-conditioned with 950 Rads (splitdose; 2 hours apart) total body X-Ray irradiation (RS 2000 Small Animal Irradiator, Rad Source Technologies Inc.) 4 hours prior to transplantation. To obtain HSCs for competitive transplantation, WBM was depleted of lineage-committed hematopoietic cells using a Lineage Cell Depletion Kit (Miltenyi Biotech, 130-110-470), stained with antibodies raised against SCA1 (D7; Biolegend), cKIT (2B8; Biolegend), CD150 (TC15-12F12.2; Biolegend), and CD48 (HM48-1; Biolegend), and HSCs (defined as DAPI-lineage-SCA1+cKIT+CD150+CD48−) were FACS sorted to purity. For primary transplantations, pre-conditioned CD45.1 recipients were injected via retro-orbital sinus with the indicated numbers of CD45.2+ donor HSCs along with CD45.1+ WBM competitor cells. Multi-lineage engraftment was monitored post-transplantation by flow cytometry analysis of RBC-lysed PB stained with antibodies raised against CD45.2 (104; Biolegend), CD45.1 (A20; Biolegend), TER119 (TER119; Biolegend), GR1 (RB6-8C5; Biolegend), CD11B (M1/70; Biolegend), B220 (RA3-6B2; Biolegend), and CD3 (17A2; Biolegend). For secondary WBM transplantations, BM cells from primary recipients were isolated and 2×106 donor cells were injected into preconditioned CD45.1 recipient mice. For secondary HSC transplantations, CD45.2+ HSCs from long-term engrafted primary transplant recipients were FACS purified, and injected along with freshly isolated CD45.1+ WBM competitor cells into preconditioned CD45.1 recipients. Multi-lineage hematopoietic engraftment was monitored by flow cytometry analysis of RBC-lysed PB as described above.

### PVA HSC Expansion

Ex vivo HSC expansion and transplantation assays were performed as described previously 47. Briefly, HSCs (defined as DAPI-lineage—SCA 1+cKIT+CD150+CD48−) were FACS sorted into fibronectin coated 96-well tissue culture plates (Corning, 354409) containing PVA-based expansion media with the following composition:
F12 Medium (11765054, LifeTech) 485 ml
ITSX Supplement 100× (51500056, LifeTech) 5 ml
Pen-Strep-Glutamax 100× (10378016, LifeTech) 5 ml
1 M HEPES (25-060-CI, Corning) 5 ml
PVA Powder (P8136, Sigma) 500 mg

After dissolution, PVA-base media was filtered (0.22 μm) and stored at 4° C. Recombinant murine SCF (PeproTech 250-03) to achieve a final concentration of 10 ng/ml and recombinant murine TPO (PeproTech 315-14) to achieve a final concentration of 100 ng/ml were added to the PVA-base media for HSC expansions.

Recombinant murine NTN1 (R&D Systems 1109-N1/CF) was added for HSC expansions with NTN1 (100 ng/ml final concentration), while a corresponding volume of PBS served as vehicle controls for expansions. Complete media exchanges were performed on Days 6, 8 and 10, and expansions were harvested on Day 11 for hematopoietic analysis and transplantations.

Expansions cells were collected by gentle mixing with a P200 pipette, followed by two serial washes of each well with ice-cold PBS to ensure complete recovery of expansion cells. Harvested cells were centrifuged in a swinging-bucket rotor at 300×g for 10 minutes at 4° C., and supernatants were gently aspirated under low suction pressure with a P10 aspirating tip. Expansion cells were stained with antibody targeting CD45, and equal numbers of DAPI-CD45+ expansion cells were FACS sorted into micro-centrifuge tubes for transplantations.

### Mitochondrial Membrane Potential Estimation

Mitochondrial membrane potential within HSCs were quantified by flow cytometry, as described previously 23. Briefly, WBM cells from femurs and tibiae were depleted of lineage committed hematopoietic progenitors and surface stained for 30 minutes at 40 C with antibodies raised against SCA1 (D7; Biolegend), cKIT (2B8; Biolegend), CD150 (TC15-12F12.2; Biolegend), and CD48 (HM48-1; Biolegend). Following surface staining, cells were washed with MACS buffer and re-suspended in micro-centrifuge tubes containing 0.5 mL DMEM with TMRE (100 nM, ThermoFisher Scientific T669) and Verapamil (50 μM, Sigma-Aldrich V4629). Cells were incubated in a 37° C. CO2 incubator for 25 minutes with caps open. Following incubation, cells were washed twice with ice-cold MACS buffer. Washed cells were re-suspended in ice-cold MACS buffer and maintained at 40 C under low-light conditions until flow cytometry acquisition. Cells incubated in DMEM without TMRE served as gating controls. TMRE sensitivity was confirmed by collapse of TMRE intensity to background levels after mitochondrial uncoupling induced by 20 μM FCCP.

### Reactive Oxygen Species Estimation

WBM cells from femurs and tibiae were depleted of lineage committed hematopoietic progenitors and surface stained for 30 minutes at 4° C. with antibodies raised against SCA1 (D7; Biolegend), cKIT (2B8; Biolegend), CD150 (TC15-12F12.2; Biolegend), and CD48 (HM48-1; Biolegend). Following surface staining, cells were washed with MACS buffer and re-suspended in micro-centrifuge tubes containing 0.5 mL DMEM with CMH2DCFDA (1 μM, Thermo Fisher Scientific C6827). Cells were incubated in a 37° C. CO2 incubator for 25 minutes with caps open. Following incubation, cells were washed twice with ice-cold MACS buffer. Washed cells were re-suspended in ice-cold MACS buffer and kept at 40 C under low-light conditions until flow cytometry acquisition.

### Cell Cycle Analysis

For HSC cell cycle analysis, WBM cells from femurs and tibiae were depleted of lineage committed hematopoietic progenitors and surface stained for 30 minutes at 4° C. with antibodies raised against SCA1 (D7; Biolegend), cKIT (2B8; Biolegend), CD150 (TC15-12F12.2; Biolegend), and CD48 (HM48-1; Biolegend). Following surface staining, cells were washed with MACS buffer, fixed and permeabilized using the BD Cytofix/Cytoperm Kit (BD Biosciences 554714) as per manufacturer's recommendations. Fixed and permeabilized cells were subsequently stained with an antibody raised against Ki67 (B56, BD561165) and counterstained with Hoechst 33342 (BD Biosciences 561908). Cells were analyzed using Flow Cytometry with a low acquisition rate (˜350 events/second). Cell cycle phases were defined as follows:
G0 (Ki−67negative; 2N DNA), G1 (Ki−67+; 2N DNA), and S/G2/M (Ki−67+; >2N DNA).

For cell cycle analysis of BMECs and LepR+ cells, femurs and tibiae were gently crushed using a mortar and pestle, enzymatically disassociated with Digestion Buffer for 15 minutes at 37° C., filtered and washed in MACS buffer and surface stained with antibodies raised against CD45 (30-F11; Biolegend), TER119 (TER119; Biolegend), CD31 (390; Biolegend), and LEPR (R&D BAF497) for 30 minutes at 4° C. Following surface staining, cells were fixed, permeabilized, and stained with an antibody raised against Ki67 and counterstained with Hoechst 33342 as described above for HSCs. BMECs were defined as CD45-TER119-CD31+, while LepR+ cells were defined as CD45−TER119−LEPR+. Cell cycle phases were defined as follows:
G0 (Ki−67negative; 2N DNA), G1 (Ki−67+; 2N DNA), and S/G2/M (Ki−67+; >2N DNA).

### Comet Assays

Comet assays were performed utilizing the CometAssayR Electrophoresis System II (R&D Systems, 4250-050-ES) as per manufacturer's recommendations. Briefly, 5000 -25000 MSCs, BMECs or HSCs were FACS sorted into 1.5 mL micro-centrifuge tubes containing 0.75 mL ice-cold PBS. Tubes were pre-marked on the outside to delineate 50 μL volume. Following FACS, tubes were centrifuged in a swinging-bucket rotor at 300×g for 10 minutes at 4° C. and supernatant was gently aspirated with low suction utilizing a P10 aspirating tip leaving behind 50 μL residual volume. 20 μL supernatant was further removed using a P20 pipette, and cells were resuspended in the residual 30 μL PBS. Cells were transferred to pre-warmed 1.5 mL micro-centrifuge tubes (37° C.) placed on a heating block, containing 300 μL molten low-melting agarose. Cells were gently mixed with agarose utilizing pre-warmed P1000 pipette tips, and 30 μL of agarose-cell suspension was transferred onto each well of the pre-warmed Comet Slide (R&D Systems, 4252-040-ESK) using pre-warmed P200 pipette tips. Slides were placed at 4° C. for 30 minutes following which they were gently immersed in Comet Assay Lysis Solution (R&D Systems, 4250-010-01) overnight at 4° C. After draining excess lysis solution, slides were placed in alkaline unwinding solution (200 mM NaOH, 1 mM EDTA, pH>13) for 1 hour at 4° C., following which electrophoresis was performed in 850 mL chilled alkaline electrophoresis solution (200 mM NaOH, 1 mM EDTA, pH>13) at 21V constant voltage for 30 minutes. Slides were sequentially washed in water and 70% ethanol, air dried, and counterstained with SYBR Gold (Thermo Fisher Scientific, S11494) solution as per manufacturer's recommendations. Images were acquired on a Ti2 epifluorescence microscope (Nikon). Image analysis was performed utilizing the CometAssayR Analysis Software (R&D Systems, 4260-000-CS) with default parameters, and all identified comets were manually reviewed for accuracy.

### Vascular Permeability (Evans Blue Dye Extravasation)

Bone marrow vascular integrity was examined as follows: 0.5% w/v Evans Blue Dye (Sigma-Aldrich E 2129) in PBS was injected via tail vein at 25 mg dye/kg total body weight. Three hours post-injection, mice were sacrificed via cervical dislocation and cardiac perfused with 10 mL PBS. Femurs denuded of tissue were crushed in a mortar and pestle with 600 μL formamide (Millipore-Sigma S4117) and incubated at 55° C. overnight. Extractions were briefly vortexed and centrifuged at 16,000×g for 5 minutes at room temperature. Supernatant was removed and absorbance (Abs) was measured at 620 nm and 740 nm. Sample Abs was corrected for heme-containing proteins [Abs620−(1.426×Abs740+0.03)] and blanked using non-injected controls [corrected sample Abs620−corrected non-injected control Abs620]. Evan's Blue Dye extravasation was calculated using a standard curve and normalized to femur weight.

### BMEC and BM LepR+ Cell Isolation

For niche cell RNA-Seq and stromal differentiation analyses, total hematopoietic cells were depleted from dissociated whole bone marrow using the EasyEights EasySep Magnet (StemCell Technologies 18103) with sheep anti-rat IgG Dynabeads (ThermoFisher Scientific 11035) pre-captured with CD45 (30-F11; Biolegend) and TER119 (TER119; Biolegend) antibodies according to the manufacturer's protocol. Briefly, femurs and tibiae were gently crushed with a mortar and pestle and enzymatically dissociated with Digestion buffer for 15 minutes at 37° C., filtered (40 μm; Corning 352340), washed in MACS buffer, and resuspended in 4 mL MACS buffer.

For bead/antibody capture, Dynabeads were prewashed with MACS Buffer and incubated for 30 minutes at 4° C. with either αCD 45 or αTER119 antibodies. To remove unbound antibody, Dynabeads were washed four times with MACS Buffer via magnetic separation and resuspended to their initial volume in MACS Buffer. 200 μL CD45-Dynabeads plus 200 μL TER119-Dynabeads were added to the digested WBM cells and incubated for 30 minutes at 4° C. with agitation. To deplete hematopoietic cells, samples were placed on the magnet for 2 minutes and resulting flow through was retained. Dynabeads were washed two additional times with 4 mL MACS Buffer to recover additional niche cells; flow through was collected after each wash, combined, and centrifuged at 500×g for 5 minutes at 4° C. to pellet hematopoietic-depleted bone marrow niche cells.

### LepR+ Cell Differentiation

To isolate LEPR+ cells for culture, WBM was isolated and depleted of hematopoietic cells as described above. Resulting cells were stained with CD45 (30-F11; Biolegend), TER119 (TER119; Biolegend), CD31 (390; Biolegend), and Biotinylated-LEPR (R&D BAF497, 4 mg/mL) for 30 minutes at 4° C. Stained cells were washed with MACS Buffer and incubated with Streptavidin-BV 421 (Biolegend 405226, 0.2 mg/mL) for 30 minutes at 4° C. Samples were washed with MACS Buffer and FACS sorted for DAPI−CD45−TER119−CD31−LEPR+ cells. 15,000 LEPR+ cells were plated in individual wells of a 96 well plate with either Osteogenic Differentiation Media (StemCell Tech 05504) or Adipogenic Differentiation Media (StemCell Tech 05507) and incubated at 37° C. 5% CO2 5% O2; media was changed according to the manufacturer's recommendations. Cells were processed following 28 days (Osteo) or 8 days (Adipo) of culture.

To quantify osteogenic differentiation, cells were washed with PBS and fixed in 4% PFA in PBS for 60 minutes at room temperature. Fixed cells were washed with molecular biology grade water and stained with 2% w/v Alizarin Red (Sigma Aldrich A5533) pH 4.1-4.3 for 60 minutes at room temperature. Following staining, cells were washed four times with molecular biology grade water.

To extract Alizarin Red for quantification, cells were incubated in 32 μL 10% v/v acetic acid for 30 minutes at room temperature with gentle agitation. Supernatant/cells were subsequently removed, vortexed for 30 sec, heated to 85° C. for 10 minutes, centrifuged at 20,000×g for 15 minutes at 4° C., and supernatant was added to 12.8 μL 10% v/v NaOH. Absorbance was read at 405 nm using the Spark Microplate Reader (TECAN).

To quantify adipogenic differentiation, BODIPY (Thermo Fisher Scientific D3922) was resuspended in DMSO according to the manufacturer's recommendations, added to cultured cells at a final concentration of 10 μM, and incubated at 37° C. 5% CO2 5% O2 for 10 minutes. Following adipocyte labeling, total cells were washed with PBS, dissociated using 35 μL Accutase Cell Detachment Solution (Biolegend 423201), and brought to a final volume of 335 μL with MACS Buffer+1 μg/mL DAPI (Biolegend 422801). BODIPY+ cells were quantified on a BD FACS Aria III using a 100 μm nozzle and 20 PSI sheath pressure.

### NTN1 Immunofluorescence

B6.129(Cg)-Leprtm2(cre)Rck/J mice were crossed with B6.Cg-Gt(ROSA)26Sortm14(CAG-tdTomato)Hze/J (JAX Stock No. 007914) mice to achieve LepR−Cre+/tdTomato+ reporter mice. To label the vasculature, mice were anesthetized and intravenously administered 10 μg anti-mouse CD144 (α-VEcadherin; Clone BV13, Biolegend) antibody. After 10 minutes, mice were euthanized and femurs isolated, stripped of muscle and connective tissue, and fixed in 4% paraformaldehyde overnight at 4° C. Bones were washed in PBS 3×5 minutes and cryo-protected in 30% Sucrose in PBS for 48 hours at 4° C. Bones were then embedded in a 1:1 mixture of O.C.T. (Tissue-Tek) and 30% Sucrose solution, and snap-frozen under liquid nitrogen. Bones were sectioned on a Leica CM 3050S cryostat at 12 μm slices, and collected on CFSA 0.5× Slides (Leica) using the Leica CryoJane tape-transfer method. Slides were brought to room temperature and washed in PBS 3×5 minutes to remove the O.C.T. Sections were delineated with an ImmEdge Pen (Vector Laboratories), blocked and permeabilized in blocking buffer (PBS containing 10% Normal Donkey Serum with 0.1% Triton X-100) for 2 hours at room temperature. Sections were stained with an antibody raised against NTN1 (R&D Systems, AF1109, 1:100 dilution), and antibodies raised against mature hematopoietic cells (αGR1/CD11B/B220/CD3/CD41/TER119) in blocking buffer and incubated overnight at 4° C. Sections were washed in PBS 3×5 minutes, and stained with donkey anti-goat secondary antibody (Thermo Fisher Scientific) in blocking buffer for 1 hour at room temperature. Slides were washed 3×5 minutes in PBS, mounted with Prolong Gold Antifade solution (Thermo Fisher Scientific P36930). 40 μm Z stack images were acquired with a Nikon C2 confocal microscope, denoised with Denoise.ai, and rendered into a maximum intensity projection (MIP) in NIS Elements.

For immunofluorescence analysis of cultured BMECs and BMS cells, cells were plated in 8-well chamber slides (Nunc Lab-Tek II CC2, 154941). Cells were washed in PBS and fixed in 4% PFA in PBS for 15 minutes at room temperature. Cells were then permeabilized in blocking buffer for 1 hour at room temperature. Cells were stained with an antibody raised against NTN1 (R&D Systems, AF1109, 1:100 dilution) in blocking buffer for 1 hour at room temperature. Cells were washed 3 times with PBS and stained with donkey anti-goat secondary antibody (Thermo Fisher Scientific) in blocking buffer for 30 minutes at room temperature. Cells were washed 3 times with PBS and counterstained with DAPI at 1 μg/mL, and mounted using Prolong Gold Antifade solution (Life Technologies). 40 μm Z stack images were acquired with a Nikon C2 confocal laser scanning microscope, denoised with Denoise.ai, and rendered into a maximum intensity projection (MIP) in NIS Elements.

### Whole-Mount Immunofluorescence

To assess oxygenation and vascular leakiness, mice were simultaneously injected with 10 μg anti-VEcadherin antibody (BV13, Biolegend), 100 mg/kg Pimonidazole HCl (Hypoxyprobe™-1, Hypoxyprobe Inc.), and 10 μg of 10,000 MW Dextran (ThermoFisher Scientific D-22910) via retroorbital injections. 10 minutes following injections, mice were euthanized, and femurs were isolated and fixed in 4% paraformaldehyde overnight at 4° C. Bones were washed in PBS 3×5 minutes, cryoprotected in 15% Sucrose in PBS for 24 hours at 4° C., and further cryo-protected in 30% Sucrose in PBS for 24 hours at 4° C. Bones were then embedded in a 1:1 mixture of O.C.T. (Tissue-Tek) and 30% Sucrose solution and snap-frozen under liquid nitrogen. Bones were then shaved longitudinally on a Leica CM 3050S cryostat to expose the bone marrow cavity. Shaved bones were washed in PBS 3×5 minutes to remove the O.C.T. Exposed bones were permeabilized in blocking buffer (PBS containing 20% Normal Goat Serum with 0.5% Triton X-100) for 2 hours at room temperature. Bones were stained with antibodies raised against Perilipin1 (Sigma P1998, 1:100), and hypoxyprobe (HP-Red549, Hypoxyprobe Inc., 1:100 dilution) in blocking buffer, and incubated for 48 hours at 4° C. Bones were washed 3×10 minutes in PBS and stained with a goat-anti-rabbit secondary antibody (Invitrogen) in blocking buffer overnight at 4° C. Bones were washed 3×10 minutes in PBS, and 40 μm Z stack images were acquired with a Nikon C2 confocal laser scanning microscope, denoised with Denoise. ai, and rendered into a maximum intensity projection (MIP) in NIS Elements. Images were analyzed using Image J software, as previously described 48. Briefly, raw images for Hypoxyprobe, Perilipin or Dextran were converted into 8-bit grayscale images, uniformly thresholded, and % of total area for each parameter was exported for analysis.

### HSC Cytospin

Following lineage cell depletion, HSCs were FACS sorted into micro-centrifuge tubes containing MACS buffer. Cytospins were carried out by applying 100-150 μL cell suspensions to slides using prewet Shandon Filter Cards, Cytoclip and Sample Chambers assembly (Thermo Scientific), according to the manufacturer's suggestions. Cytospins were performed using a Shandon Cytospin 4 centrifuge (Thermo Fisher Scientific) with low acceleration at 800 rpm for 3 minutes. Slides were subsequently air dried for 20 minutes, fixed for 10 minutes in 4% paraformaldehyde in PBS, rinsed three time for 5 minutes in PBS, and incubated in blocking buffer (PBS with 10% Normal Goat Serum and 0.2% Triton X-100) for 30 minutes at room temperature. Slides were then incubated in primary antibody raised against phospho-H2AX (Ser139) (JBW301; Millipore) in antibody dilution buffer (PBS with 1% BSA and 0.2% Triton X-100) overnight at 4° C. Cells were washed three times in PBS and incubated in antibody dilution buffer for 1 hour with goat anti-mouse IgG (Invitrogen A-11029, 1:1000 dilution). Slides were washed three times in PBS, stained with DAPI (Biolegend) at 1 μg/mL in PBS for 5 minutes at room temperature. Slides were washed three additional times and mounted in ProLong Gold Antifade (Thermo Fisher Scientific Scientific). Cells were imaged on a Nikon C2 confocal microscope. Images were analyzed in Image J as previously described for quantifying nuclear proteins 49. A minimum of 500 HSCs were analyzed in aged mice treated with PBS or NTN1.

RNA isolation

For HSC RNA isolation, lineage cell depletion was performed as described above and stained with antibodies raised against SCA1 (D7; Biolegend), cKIT (2B8; Biolegend), CD150 (TC15-12F12.2; Biolegend), and CD48 (HM48-1; Biolegend) and HSCs (defined as DAPI-lineage—SCA1+cKIT+CD150+CD48−) were FACS sorted directly into TRIzol LS Reagent (Thermo Fisher Scientific 10296010).

For BMEC and LepR+ cell RNA isolation, mice were intravitally labeled with an antibody raised against VEcadherin (BV13; Biolegend) 10 minutes prior to sacrifice. Femurs and tibiae were gently crushed using a mortar and pestle and enzymatically dissociated with Digestion buffer for 15 minutes at 37° C., filtered, washed in MACS buffer, and depleted of terminally differentiated hematopoietic cells as described under ‘BMEC and BM LepR+ cell isolation’.

Depleted cell suspensions were stained with antibodies raised against CD45 (30-F11; Biolegend), TER119 (TER119; Biolegend), CD31 (390; Biolegend), and LEPR (R&D BAF497). Stained cells were washed in MACS buffer. BMECs (defined as CD45-TER119-Vecad+CD31+) and LepR+ cells (defined as CD45−TER119−VEcadherin−LEPR+) were sorted directly into TRIzol LS Reagent. RNA was purified from TRIzol LS according to manufacturer's recommendations.

### RNA Sequencing and Mapping

RNA concentration was estimated using the Agilent High Sensitivity RNA Screen Tape System, and libraries were prepared using the SMART-SeqR v4 UltraR Low Input Kit (Takara Bio) with 5 ng total RNA input per sample, as per manufacturer recommendations. cDNA libraries were subject to high throughput sequencing (Illumina NovaSeq) and ˜50 million paired-end reads were generated per sample. Reads were checked for quality (FastQC v0.11.5) and processed using the Digital Expression Explorer 2(DEE 2 ) workflow 50. Adapter trimming was performed with Skewer (v0.2.2) 51.

Further quality control was performed with Minion, part of the Kraken package 52. Filtered reads were mapped to the mouse reference genome GRCm38 using STAR aligner 53 and gene-wise expression counts were generated using the “-quantMode GeneCounts” parameter. The R package edgeR was used to calculate FPKM 54.

### RNA-Seq Analysis

Normalized read counts were uploaded and analyzed using the integrated Differential Expression and Pathway (iDEP) (http://bioinformatics.sdstate.edu/idep94/) analysis pipeline 55. For BMEC and LEPR+ cell RNA-Seq analysis (N=5 samples per group), normalized read counts were uploaded to iDEP, annotated with mouse ENSEMBL Gene IDs, and genes with low expression values (FPKM<0.5 in 5 samples) were filtered out with default settings for normalization (Constant c for started log: log(x+c)=1). 12121 genes (LepR+ cells; LepR-NTN1 mice), 11397 genes (BMECs; LepR-NTN1 mice), 11923 genes (LepR+ cells; CDH5-NTN1 mice) and 11441 genes (BMECs; CDH5-NTN1 mice) passed the filter. Filtered and processed datasets were downloaded and utilized for performing GSEA. Heatmaps depicting unsupervised hierarchical clustering of the Top 500 differentially expressed genes were generated with default parameters (Distance: Correlation, Linkage: Average, Cutoff Z score: 4, Center Genes: Subtract Mean). The numbers of differentially expressed genes were estimated with the cutoffs for Log2 Fold Change>1.25 and False Discovery Rate (FDR) <0.1. For HSC RNA-Seq analysis (N=3 Samples per group, N=3 Groups), genes with low expression values were filtered out with a more stringent cut-off (FPKM<1 in 3 samples), owing to smaller number of samples per group, with default settings for normalization. Heatmaps depicting unsupervised hierarchical clustering of the Top 100 differentially expressed genes were generated with default parameters. For pairwise comparisons (Young vs Aged PBS and Aged PBS vs Aged NTN1), similar FPKM cutoffs (FPKM<1 in 3 samples) was utilized to filter out low-abundant genes, and the processed datasets (N=12717 genes for Young vs Aged PBS, N=12256 genes for Aged PBS vs Aged NTN1) that passed filter were downloaded and utilized for GSEA.

### GSEA

For performing GSEA of niche cells and HSCs, GSEA input files were created using filtered and processed datasets downloaded from iDEP. Briefly, genes that were not annotated with ENSEMBL IDs (˜50 genes/dataset) were excluded. For the remaining genes, Log2 FPKM were converted to FPKM, and expression dataset files (.GCT) and phenotype label files (.CLS) were generated in Microsoft Excel. GSEA was performed with the following default parameters:
Number of permutations: 1000
Collapse to Gene Symbols
Permutation Type: Gene Set (Less than 7 samples per group).
Chip Platform: Human_ENSEMBL_GENE_ID.MSigDB_V7.4.Chip
Gene Set Size: 15-500
GSEA was performed utilizing the HALLMARK (V7.4), REACTOME (V7.4) collections and DNA
Repair pathways in the KEGG (V7.4) database.

### HSC Aging Signature

Genes identified as differentially expressed in the ‘Aging List Reanalysis’ (Consistency Score≥2) were obtained from Supplemental Table 2 associated with the Svendsen et al manuscript 22. The analysis identified 1131 genes that were consistently altered with age (reported in at least 2 published datasets), of which 749 were upregulated and 382 were down regulated genes. After removing unannotated genes, 717 uniquely upregulated genes and 366 uniquely down regulated genes were identified. The Top 500 upregulated genes (based on Consistency Score and Fold Change), and 366 down regulated genes were utilized to create the ‘Svendsen Aged’ and ‘Svendsen Young’ comprehensive HSC signatures, respectively.

### Pre-ranked GSEA

To identify pathways that are consistently altered during HSC aging, Pre-Ranked GSEA analysis was performed on the comprehensive HSC aging signature (Svendsen et al). 1131 genes that were consistently altered with age, were ranked by multiplying Log2 Fold Change with their Consistency Score. Gene names were converted Human Gene symbols. Unannotated genes were filtered out, and the remaining ranked list of genes (1079 genes) was analyzed by performing Pre-Ranked GSEA with the following default parameters:
Number of permutations: 1000
No Collapse/Remap
Permutation Type: Gene Set
Chip Platform: NA
Gene Set Size: 15-500
GSEA was performed utilizing the HALLMARK (V7.4), REACTOME (V7.4) collections and DNA Repair pathways in the KEGG (V7.4) database.

### Statistics

Sample sizes for phenotypic and functional analysis of mouse hematopoietic parameters were determined based on estimates of variance and effect sizes determined in previous experiments. Number of animals needed were calculated based on the ability to detect a two-fold change in the Mean with 80% power, with the threshold for significance (a) set at 0.05. Both male and female mice were utilized for experiments, and a similar proportion of genders across experimental groups was maintained in all experiments.

All experimental findings including transplantations were confirmed in at least 2 independent cohorts of mice, and the data presented represent pooled data from independent experiments. Statistical comparisons between two groups were performed using two-tailed Student's t-test. Multiple comparisons were performed using One-way ANOVA analysis with a Tukey's Correction. Data is presented as the mean and standard error of the mean (SEM), unless otherwise noted. Statistical significance is indicated as * (p<0.05), ** (p<0.01), *** (p<0.001), and n.s. (not significant). Statistical analysis was performed using Prism 6 (GraphPad Software).

Any headings and sub-headings utilized in this description are not meant to limit the embodiments described thereunder. Features of various embodiments described herein may be utilized with other embodiments even if not described under a specific heading for that embodiment.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

### REFERENCES

1 Ho, Y. H. & Mendez-Ferrer, S. Microenvironmental contributions to hematopoietic stem cell aging. Haematologica 105, 38-46, doi:10.3324/haematol.2018.211334 (2020).
2 Oh, J., Lee, Y. D. & Wagers, A. J. Stem cell aging: mechanisms, regulators and therapeutic opportunities. Nature medicine 20, 870-880, doi:10.1038/nm.3651 (2014).
3 Rosko, A. E. et al. Advances in Management for Older Adults With Hematologic Malignancies. J Clin Oncol 39, 2102-2114, doi:10.1200/jco.21.00242 (2021).
4 Rosko, A. & Artz, A. Aging: Treating the Older Patient. Biol Blood Marrow Transplant 23, 193-200, doi:10.1016/j. bbmt.2016.11.007 (2017).
5 Balducci, L. Myelosuppression and its consequences in elderly patients with cancer. Oncology (Williston Park) 17, 27-32 (2003).
6 Hooper, A. T. et al. Engraftment and reconstitution of hematopoiesis is dependent on VEGFR2-mediated regeneration of sinusoidal endothelial cells. Cell stem cell 4, 263-274, doi:S1934-5909(09)00009-5 [pii] 10.1016/j.stem.2009.01.006 (2009).
7 Crane, G. M., Jeffery, E. & Morrison, S. J. Adult haematopoietic stem cell niches. Nature reviews. Immunology 17, 573-590, doi:10.1038/nri.2017.53 (2017).
8 Ramalingam, P., Butler, J. M. & Poulos, M. G. Vascular Regulation of Hematopoietic Stem Cell Homeostasis, Regeneration, and Aging. Current stem cell reports 7, 194-203, doi:10.1007/s40778-021-00198-2 (2021).
9 Ramalingam, P. et al. Endothelial mTOR maintains hematopoiesis during aging. The Journal of experimental medicine 217, doi:10.1084/jem.20191212 (2020).
10 Boyer, N. P. & Gupton, S. L. Revisiting Netrin-1: One Who Guides (Axons). Front Cell Neurosci 12, 221, doi:10.3389/fncel.2018.00221 (2018).
11 Castets, M. & Mehlen, P. Netrin-1 role in angiogenesis: to be or not to be a pro-angiogenic factor? Cell Cycle 9, 1466-1471, doi:10.4161/cc.9.8.11197 (2010).
12 Maruyama, K., Takemura, N., Martino, M. M., Kondo, T. & Akira, S. Netrins as prophylactic targets in skeletal diseases: A double-edged sword? Pharmacol Res 122, 46-52, doi:10.1016/j.phrs.2017.05.011 (2017).
13 Renders, S. et al. Niche derived netrin-1 regulates hematopoietic stem cell dormancy via its receptor neogenin-1. Nat Commun 12, 608, doi:10.1038/s41467-020-20801-0 (2021).
14 Gulati, G. S. et al. Neogenin-1 distinguishes between myeloid-biased and balanced Hoxb5(+) mouse long-term hematopoietic stem cells. Proceedings of the National Academy of Sciences of the United States of America 116, 25115-25125, doi:10.1073/pnas.1911024116 (2019).
15 Helbling, P. M. et al. Global Transcriptomic Profiling of the Bone Marrow Stromal Microenvironment during Postnatal Development, Aging, and Inflammation. Cell reports 29, 3313-3330 e3314, doi:10.1016/j.celrep.2019.11.004 (2019).
16 Bin, J. M. et al. Complete Loss of Netrin-1 Results in Embryonic Lethality and Severe Axon Guidance Defects without Increased Neural Cell Death. Cell reports 12, 1099-1106, doi:10.1016/j.celrep.2015.07.028 (2015).
17 Ding, L., Saunders, T. L., Enikolopov, G. & Morrison, S. J. Endothelial and perivascular cells maintain haematopoietic stem cells. Nature 481, 457-462, doi:10.1038/nature 10783 (2012).
18 Sorensen, I., Adams, R. H. & Gossler, A. DLL1-mediated Notch activation regulates endothelial identity in mouse fetal arteries. Blood 113, 5680-5688, doi:10.1182/blood-2008-08-174508 (2009).
19 Poulos, M. G. et al. Endothelial transplantation rejuvenates aged hematopoietic stem cell function. The Journal of clinical investigation 127, 4163-4178, doi:10.1172/JCI93940 (2017).
20 Inomata, K. et al. Genotoxic stress abrogates renewal of melanocyte stem cells by triggering their differentiation. Cell 137, 1088-1099, doi:10.1016/j.cell.2009.03.037 (2009).
21 Ren, B. et al. E2F integrates cell cycle progression with DNA repair, replication, and G(2)/M checkpoints. Genes Dev 16, 245-256, doi:10.1101/gad.949802 (2002).
22 Flohr Svendsen, A. et al. A comprehensive transcriptome signature of murine hematopoietic stem cell aging. Blood 138, 439-451, doi:10.1182/blood.2020009729 (2021).
23 Mansell, E. et al. Mitochondrial Potentiation Ameliorates Age-Related Heterogeneity in Hematopoietic Stem Cell Function. Cell stem cell 28, 241-256.e246, doi:10.1016/j.stem. 2020.09.018 (2021).
24 Chambers, S. M. et al. Aging hematopoietic stem cells decline in function and exhibit epigenetic dysregulation. PLoS Biol 5, e201, doi:06-PLBI-RA-2319 [pii] 10.1371/journal. pbio.0050201 (2007).
25 Sun, D. et al. Epigenomic profiling of young and aged HSCs reveals concerted changes during aging that reinforce self-renewal. Cell stem cell 14, 673-688, doi:10.1016/j.stem.2014.03.002 (2014).
26 Beerman, I., Seita, J., Inlay, M. A., Weissman, I. L. & Rossi, D. J. Quiescent hematopoietic stem cells accumulate DNA damage during aging that is repaired upon entry into cell cycle. Cell stem cell 15, 37-50, doi:10.1016/j.stem.2014.04.016 (2014).
27 Flach, J. et al. Replication stress is a potent driver of functional decline in ageing haematopoietic stem cells. Nature 512, 198-202, doi:10.1038/nature13619 (2014).
28 Ho, T. T. et al. Aged hematopoietic stem cells are refractory to bloodborne systemic rejuvenation interventions. The Journal of experimental medicine 218, doi:10.1084/jem.20210223 (2021).
29 Chen, C., Liu, Y. & Zheng, P. mTOR regulation and therapeutic rejuvenation of aging hematopoietic stem cells. Science signaling 2, ra75, doi:2/98/ra75 [pii] 10.1126/scisignal.2000559 (2009).
30 Florian, M. C. et al. Cdc42 activity regulates hematopoietic stem cell aging and rejuvenation. Cell stem cell 10, 520-530, doi:10.1016/j.stem.2012.04.007 (2012).
31 Mohrin, M. et al. Stem cell aging. A mitochondrial UPR-mediated metabolic checkpoint regulates hematopoietic stem cell aging. Science 347, 1374-1377, doi:10.1126/science.aaa2361 (2015).
32 Luo, H. et al. Mitochondrial Stress-Initiated Aberrant Activation of the NLRP3 Inflammasome Regulates the Functional Deterioration of Hematopoietic Stem Cell Aging. Cell reports 26, 945-954 e944, doi:10.1016/j.celrep.2018.12.101 (2019).
33 Chang, J. et al. Clearance of senescent cells by ABT263 rejuvenates aged hematopoietic stem cells in mice. Nature medicine 22, 78-83, doi:10.1038/nm.4010 (2016).
34 Valletta, S. et al. Micro-environmental sensing by bone marrow stroma identifies IL-6 and TGFbeta1 as regulators of hematopoietic ageing. Nat Commun 11, 4075, doi:10.1038/s41467-020-17942-7 (2020).
35 Young, K. et al. Decline in IGF1 in the bone marrow microenvironment initiates hematopoietic stem cell aging. Cell stem cell 28, 1473-1482 e1477, doi:10.1016/j.stem.2021.03.017 (2021).
36 Maryanovich, M. et al. Adrenergic nerve degeneration in bone marrow drives aging of the hematopoietic stem cell niche. Nature medicine, doi:10.1038/s41591-018-0030-x (2018).
37 Keyes, B. E. & Fuchs, E. Stem cells: Aging and transcriptional fingerprints. J Cell Biol 217, 79-92, doi:10.1083/jcb.201708099 (2018).
38 Niedernhofer, L. J. et al. Nuclear Genomic Instability and Aging. Annu Rev Biochem 87, 295-322, doi:10.1146/annurev-biochem-062917-012239 (2018).
39 Yousefzadeh, M. et al. DNA damage—how and why we age? Elife 10, doi:10.7554/eLife.62852 (2021).
40 Hoeijmakers, J. H. DNA damage, aging, and cancer. N Engl J Med 361, 1475-1485, doi:10.1056/NEJMra0804615 (2009).
41 Schumacher, B., Pothof, J., Vijg, J. & Hoeijmakers, J. H. J. The central role of DNA damage in the ageing process. Nature 592, 695-703, doi:10.1038/s41586-021-03307-7 (2021).
42 Brett, J. O. et al. Exercise rejuvenates quiescent skeletal muscle stem cells in old mice through restoration of Cyclin D1. Nat Metab 2, 307-317, doi:10.1038/s42255-020-0190-0 (2020).
43 Jackson, S. P. & Bartek, J. The DNA-damage response in human biology and disease. Nature 461, 1071-1078, doi:10.1038/nature08467 (2009).
44 Benedito, R. et al. The notch ligands D114 and Jagged1 have opposing effects on angiogenesis. Cell 137, 1124-1135, doi:10.1016/j. cell.2009.03.025 (2009).
45 Poulos, M. G. et al. Vascular Platform to Define Hematopoietic Stem Cell Factors and Enhance Regenerative Hematopoiesis. Stem Cell Reports 5, 881-894, doi:10.1016/j.stemcr.2015.08.018 (2015).
46 Ramalingam, P. et al. Chronic activation of endothelial MAPK disrupts hematopoiesis via NFKB dependent inflammatory stress reversible by SCGF. Nat Commun 11, 666, doi:10.1038/s41467-020-14478-8 (2020).
47 Wilkinson, A. C. et al. Long-term ex vivo haematopoietic-stem-cell expansion allows nonconditioned transplantation. Nature 571, 117-121, doi:10.1038/s41586-019-1244-x (2019).
48 Aguilera, K. Y. & Brekken, R. A. Hypoxia Studies with Pimonidazole in vivo. Bio Protoc 4, doi:10.21769/bioprotoc.1254 (2014).
49 Wessel, A. W. & Hanson, E. P. A method for the quantitative analysis of stimulation-induced nuclear translocation of the p65 subunit of NF-κB from patient-derived dermal fibroblasts. Methods Mol Biol 1280, 413-426, doi:10.1007/978-1-4939-2422-6_25 (2015).
50 Ziemann, M., Kaspi, A. & El-Osta, A. Digital expression explorer 2: a repository of uniformly processed RNA sequencing data. Gigascience 8, doi:10.1093/gigascience/giz022 (2019).
51 Jiang, H., Lei, R., Ding, S. W. & Zhu, S. Skewer: a fast and accurate adapter trimmer for next generation sequencing paired-end reads. BMC Bioinformatics 15, 182, doi:10.1186/1471-2105-15-182 (2014).
52 Davis, M. P., van Dongen, S., Abreu-Goodger, C., Bartonicek, N. & Enright, A. J. Kraken: a set of tools for quality control and analysis of high-throughput sequence data. Methods 63, 41-49, doi:10.1016/j.ymeth.2013.06.027 (2013).
53 Dobin, A. et al. STAR: ultrafast universal RNA-seq aligner. Bioinformatics 29, 15-21, doi:10.1093/bioinformatics/bts635 (2013).
54 Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
55 Ge, S. X., Son, E. W. & Yao, R. iDEP: an integrated web application for differential expression and pathway analysis of RNA-Seq data. BMC Bioinformatics 19, 534, doi:10.1186/s12859-018-2486-6 (2018).

1. A pharmaceutical composition comprising a therapeutic drug containing an effective dose of netrin-1 (NTN-1) in a pharmaceutically acceptable carrier or vehicle, wherein the netrin-1 is sufficient to reactivate DNA Damage Response (DDR), resolve DNA damage, and restore functional potential within an aged bone marrow niche and blood stem cells and restore regenerative capacity of an aged hematopoietic system to endure serial chemotherapy regimens.

2. The pharmaceutical composition according to claim 1, wherein the therapeutic drug is given as a systemic infusion administration to a patient.

3. The pharmaceutical composition according to claim 1, wherein treatment of acute myeloid leukemia (AML) with the NTN1 results in a decrease in the expansion of AML cells due to apoptosis.

4. The pharmaceutical composition according to claim 3, wherein the NTN1 antagonizes leukemia by maintaining aged vascular endothelium (VE) and mesenchymal stem cells (MSC) integrity to maintain aged hematopoietic stem cells (HSC), while eradicating AML simultaneously.

5. The pharmaceutical composition according to claim 1, wherein mesenchymal stem cells (MSC) that are supplemented in an ex vivo expansion protocol with the NTN1 maintain and enhance function of expanded young and aged hematopoietic stem cells (HSC) for gene correction of hemaglobinopathies.

6. The pharmaceutical composition according to claim 1, wherein systemic infusion of NTN1 restores aged hematopoietic stem cells (HSC) function by reactivating the DNA Damage Response (DDR), and the NTN1 reactivates DDR to promote rapid hematopoietic recovery, improved self-renewal capacity, increased longevity, and reduces weight loss following single and serial myelosuppressive insults as compared to non-treatment with the therapeutic drug.

7. Use of a therapeutic drug which contains an effective dose of netrin-1(NTN-1) for treatment of hemoglobinopathies.

8. The use according to claim 7, wherein the hemoglobinopathies includes thalassemia

9. The use according to claim 7, wherein the NTN1 is able to induce reactivation of DNA damage response (DDR) and rejuvenation of an aged hematopoietic system.

10. A method of using a pharmaceutical composition comprising:
administrating a therapeutic drug containing an effective dose of netrin-1 (NTN-1) to a patient for providing a NTN1-treatment; and
using the NTN1-treatment in preserving a bone marrow (BM) niche for promoting stem cell health as compared to not providing the NTN1-treatment.

11. The method of claim 10, wherein the therapeutic drug is for hematological malignancies and minimizes toxicities associated with strand of care myelosuppressive treatments as compared to conventional treatment.

12. The method of claim 10, further comprises utilizing Recombinant murine NTN1 protein reconstituted in PBS at 100 μg/mL.

13. The method of claim 12, further comprises taking a dosage of 4 μg NTN1; diluting the dosage of 4 μg NTN-1 to a final volume of 100 μL; and injecting the final volume subcutaneously.

14. The method of claim 13, wherein the injecting the final volume negatively affects acute myeloid leukemia (AML) aggressiveness.

15. The method of claim 14, wherein the injecting the final volume rejuvenates stem cells function of supportive niche within bone marrow of mice.

16. The method of claim 15, wherein the injecting the final volume further includes preserving a hematopoietic/vascular function while simultaneously targeting AML cells.

17. The method of claim 10, further comprises performing alkaline comet assays and confirming that the NTN1-treatment resolves DNA damage in aged HSCs to levels observed in young mice.

18. The method of claim 17, further comprises demonstrating that the NTN1-treatment reactivates DNA damage response (DDR) and resolving DNA damage within aged hematopoietic stem cells (HSCs).

19. The method of claim 18, further comprises maintaining HSC homeostatis as compared to not having the NTN1-treatment.

20. The method of claim 10, further comprises utilizing the NTN1-treatment as a therapeutic modality that reverses age-related hematopoietic deficiencies while simultaneously targeting growth and survival of acute myelogenous leukemia (AML) as compared to not utilizing the NTN1-treatment.
